(19) United States
(12) Patent Application Publication
(10) Pub. No.: US 2021/0278393 A1
(43) Pub. Date: Sep. 9, 2021

(54) METHOD FOR MEASURING SINGLE-CELL BIOMASS TO PREDICT CLINICAL OUTCOMES FOR STEM CELL TRANSPLANT PATIENTS

(71) Applicant: VIRGINIA COMMONWEALTH UNIVERSITY, Richmond, VA (US)

(72) Inventors: Jason REED, Richmond, VA (US); Amir TOOR, Richmond, VA (US); Kevin LESLIE, Richmond, VA (US)

(73) Assignee: Virginia Commonwealth University, Richmond, VA (US)

(21) Appl. No.: 16/330,554

(22) PCT Filed: Sep. 19, 2017
(86) PCT No.: PCT/US2017/052148

§ 371 Date: Mar. 5, 2019

Related U.S. Application Data
(60) Provisional application No. 62/396,536, filed on Sep. 19, 2016.

(51) Int. Cl. G01N 33/50; G01N 21/45

(52) U.S. Cl. CPC G01N 33/505; G01N 33/5011; G01N 2201/08; G01N 2800/50; G01N 2201/06113; G01N 21/45

(57) ABSTRACT

Provided herein are methods of determining the mass of T cells in stem cell transplant (SCT) recipients at risk of developing graft vs host disease (GVHD), and tumor (e.g. melanoma) cells in patients at risk of developing drug resistant tumors, using Live-Cell Interferometry (LCI) so that treatment may be appropriately modulated. High Speed LCI (HSLCI) apparatuses to conduct the cell mass measurements are also provided.

### CROSS-REFERENCE TO RELATED APPLICATIONS

This application claims benefit of U.S. provisional patent applications 62/396,536, filed Sep. 19, 2016, the complete contents of which is hereby incorporated by reference.

### STATEMENT OF FEDERALLY SPONSORED RESEARCH AND DEVELOPMENT

This invention was made with government support under RO1CA185189 awarded by the National Cancer Institute. The United States government has certain rights in the invention.

### BACKGROUND OF THE INVENTION

### Field of the Invention

The invention generally relates to improved methods of measuring the mass of a group of cells of interest that are associated with a disease or condition of interest, and adjusting treatment protocols in response to the measurements. In particular, in exemplary embodiments the invention provides methods of determining the mass of T cells in stem cell transplant (SCT) recipients at risk of developing graft vs host disease (GVHD), and tumor (e.g. melanoma) cells in patients at risk of developing drug resistant tumors, using Live-Cell Interferometry (LCI) so that treatment may be appropriately modulated. High Speed LCI (HSLCI) apparatuses to conduct the cell mass measurements are also provided.

### Background

Many diseases are characterized by changes in cell morphology, motion, and mechanical rigidity. Unfortunately, depending on the type of cell being considered, it typically takes from several hours to several days to detect such changes, delaying the application of suitable therapeutic responses. Two examples of diseases/conditions the treatment of which is hampered by the lack of rapid assays are graft-versus-host disease (GVHD), which can be a major complication of stem cell transplants, and the development of drug resistance in the treatment of patients with melanoma.

With respect to GVHD, approximately 32,000 allogeneic stem cell transplants (SCTs) from HLA-matched donors are performed annually. In addition to an increased susceptibility to infection due to immunosuppressive therapy to prevent rejection, recipients of these transplants face competing risks of malignancy relapse and graft versus host disease (GVHD), the incidence ranging from about 10% to 50% for allogeneic SCT recipients. In addition, an increasing number of SCTs are performed each year using matched unrelated donors (MUDs) and increasingly older patients now undergo transplantation, increasing the risk of GVDH. Many studies have sought to identify post-transplant prognostic and diagnostic biomarkers for both acute and chronic GVHD. Despite relatively inexpensive detection techniques and encouraging results, such studies have yet to yield an actionable marker or panel of markers for GVHD in the clinic.

With respect to melanoma, an estimated 87,000 new cases of melanoma will be diagnosed in the United States in 2017, with 9,700 deaths attributable to the disease. Despite comprising only 1-2% of total skin cancer diagnoses, melanoma is responsible for 75% of total skin cancer mortality. This disease is characterized by significant inter-patient, intra-patient, and even intra-tumor heterogeneity in diagnosed individuals, with mutations in the BRAF, RAS, KRAS, HRAS, AKT, and PTEN genes having been identified. The majority of these mutations lead to increased signaling in the MAPK pathway, resulting in the advent of therapeutics targeting MAPK-activating mutations (e.g. V600E and V600K in BRAF). As a result, progression-free survival (PFS) has greatly improved. Unfortunately, treatment failure occurs in most cases where inhibitor monotherapy is employed, often due to acquired mutations driving the re-activation of the MAPK pathway. To reduce the frequency of resistance, many treatment regimens prescribe a combination of inhibitors. However, resistance eventually develops in approximately 80% of patients receiving combination therapy. The genetic heterogeneity underlying the mechanisms of innate and acquired resistance makes current screening procedures incompletely predictive of drug susceptibility, both prior to, during and after therapy and the development of resistance, increasing the difficulty of selecting appropriate therapies.

It would be beneficial to have available methodology to rapidly, accurately, and inexpensively

i) predict whether or not a transplant patient is at risk for developing GVHD; and

ii) assess drug susceptibility of patients both before, during and after targeted treatment of melanoma; so that treatment regimens for patients can be adjusted/optimized as needed on an individual basis.

United States patent application 20140178865 (Reed et al.), the complete contents of which is herein incorporated by reference) discloses live cell interferometry (LCI) for rapid, real-time quantification of cell mass in cells. However, neither GVDH nor melanoma were studied or mentioned in this application and the LCI method was not high-speed LCI.

Reed et al. (Biophysical Journal, 101 (2011), 1025-1031) discloses live cell interferometry (LCI) for rapid, real-time quantification of cell mass in multiple myeloma cells.

### SUMMARY OF THE INVENTION

Other features and advantages of the present invention will be set forth in the description of invention that follows, and in part will be apparent from the description or may be learned by practice of the invention. The invention will be realized and attained by the devices and methods particularly pointed out in the written description and claims hereof.

The invention provides methods for rapidly, accurately and inexpensively i) assessing the drug susceptibility of patients before, during and after targeted treatment of cancer; and for ii) predicting whether or not a stem cell transplant patient is at risk for developing GVHD. For both diseases/conditions, the technique of Live-Cell Interferometry (LCI) is employed to assess the status of the patient, and the results are used to adjust therapeutic treatments of the patients. While LCI is employed for each disease or condition, the particular ways in which this measurement technique is used and the protocols that are involved are unique to each disease/condition. In addition, new LCI devices are provided as are methods of using the devices. The new devices enable high speed LCI (HSLCI) by incorporating a laser autofocus element, using an optical beam deflection technique, into the apparatuses.

It is an object of this invention to provide a method for predicting the risk of and prophylactically treating graft-versus-host disease (GVHD) in a subject in need thereof, comprising the steps of: measuring the cell biomass of CD3+ T cells isolated from a biological sample obtained from said subject; comparing the measured cell biomass to a reference value obtained from a control subject not at risk for GVHD; determining that said subject is at risk for developing GVHD when the measured cell biomass is higher than the reference value; and administering an immunosuppressant therapy to said subject determined to be at risk for developing GVHD.

Also provided is a method of detecting resistance to at least one anticancer drug in a subject in need thereof and treating the subject accordingly, comprising i) administering the at least one anticancer drug to the subject; ii) performing HSLCI to measure a cell biomass value of tumor cells isolated from a biological sample obtained from the subject; iii) comparing the tumor cell biomass value to a reference tumor cell biomass value obtained from a control group of tumor cells that are not resistant to the at least one anticancer drug; iv) determining that the subject is resistant to the at least one anticancer drug when the cell biomass value is greater than the reference cell biomass value or determining that the subject is not resistant to the at least one anticancer drug when the cell biomass value is less than or equal to the reference cell biomass value; and v) discontinuing administration of the at least one anticancer drug to the subject when the subject is determined in step iv) to be resistant to the at least one anticancer drug or continuing administration of the at least one anticancer drug to the subject when the subject is determined in step iv) to not be resistant to the at least one anticancer drug.

Further aspects provide a method of detecting resistance to at least one melanoma drug in a subject in need thereof and treating the subject accordingly, comprising

i) administering the at least one melanoma drug to the subject; ii) measuring a cell biomass value of tumor cells isolated from a biological sample obtained from the subject; iii) comparing the tumor cell biomass value to a reference tumor cell biomass value obtained from a control group of tumor cells that are not resistant to the at least one melanoma drug; iv) determining that the subject is resistant to the at least one melanoma drug when the cell biomass value is greater than the reference cell biomass value or determining that the subject is not resistant to the at least one melanoma drug when the cell biomass value is less than or equal to the reference cell biomass value; and v) discontinuing administration of the at least one melanoma drug to the subject when the subject is determined in step iv) to be resistant to the at least one melanoma drug or continuing administration of the at least one melanoma drug to the subject when the subject is determined in step iv) to not be resistant to the at least one melanoma drug.

The invention also provides a method of selecting and administering one or more drugs or combination of drugs for treatment of a patient having a cancer that is resistant to one or more previously administered drugs, comprising I) identifying a group of candidate drugs or combination of drugs that differ from the one or more previously administered drugs, II) performing an HSLCI analysis of a biological sample having resistant cancer cells obtained from the patient to obtain a plurality of dose response curves for each candidate drug or combination of drugs in the group of candidate drugs or combination of drugs, wherein the plurality of dose response curves is obtained at i) a highest concentration of each candidate drug or combination of drugs equal to a peak serum concentration tolerated by patients, and ii) one or more lower concentrations of each candidate drug or combination of drugs, each of which is lower than the highest concentration; III) selecting for administration a drug or combination of drugs that inhibits growth of the resistant cancer cells at the lowest of the one or more lower concentrations that are tested; and IV) administering the one or more drugs or combinations of drugs to the patient.

Further provided is a high-speed live-cell interferometry (HSLCI) apparatus, comprising

i) a camera with a positionable camera objective; ii) a quantitative phase detector; ii) a moveable observation chamber for containing a cell sample; iii) an autofocus component configured to continuously maintain the sample within an operational depth of focus of the microscope objective, wherein the autofocus component comprises a) a light source; b) a microcontroller feedback loop configured to receive a positional signal transmitted from the camera objective and calculate and transmit an offset signal; c) a precision actuator configured to receive the offset signal from the microcontroller feedback loop and adjust a position of the microscope objective in response to the offset signal; and
d) an electronic system configured to coordinate motion of the moveable observation chamber and light emitted from the light source; and iv) at least one processor configured to process phase images obtained from the camera.

Further aspects provide a live cell interferometric measurement apparatus, comprising: a means to image cells in a volume; means to compute cell mass based on images of cells; and a laser autofocus feature for focusing the imaging means on the cells in the volume.

### BRIEF DESCRIPTION OF THE DRAWINGS

FIG. 1. Overview of methods. Immunomagnetic beads were used to isolate CD3+ T cells from patient whole blood or donor apheresis product samples. Images of isolated cells were collected using our automated LCI system. Briefly, a specialized camera (Phasics, SID4BIO, France) measured local phase shifts as light (670 nm wavelength) from a fiber-coupled LED passed through the cells, enabling the non-invasive quantification of dry cell mass. For all cells in each sample, phase and standard brightfield images were collected at 40× magnification (Nikon Plan Fluor, NA 0.75, #MRH00401, Japan) under standard cell culture conditions (37° C., 5% CO₂) using phase (Phasics SID4BIO, France) and color (Basler ac645, USA) cameras, respectively. Phase images were processed using custom MATLAB programs designed to track individual cells between successive images and calculate their dry masses, yielding thousands of single-cell mass measurements at multiple time points, post-transplant.

FIG. 2. Three-dimensional rendering of patient-derived CD3+ T cells from LCI image. Increasing height and heat map color correspond to increasing mass-density in cells. Image was generated using Vision64® Operation and Analysis Software (Bruker, 2012).

FIGS. 3A and B. Example of single-cell tracking stability over time. A. Normalized mass plot of a single CD3+ T cell over 250 minutes. B. Representative LCI image at a single point in time; box highlights cell whose mass was plotted in A.

FIGS. 4A and B. Boxplots showing normalized T cell population mass distributions for two representative allogeneic transplant recipients. Product, Day 30, Day 60, and Day 100 mass measurements were normalized to the median mass of the patients' product samples. All individual cell masses measured at a given time point have been overlaid onto that time point's boxplot. A. Patient did not develop clinical signs of GVHD during the course of the study. Median CD3+ T cell mass increased at day 30, but decreased and stabilized through days 60 and 100. B. Patient developed severe chronic GVHD involving the lungs 151 days post-transplant. Median CD3+ T cell mass increased 44% above the product sample median at day 30, and remained elevated through day 100.

FIG. 5. Average normalized median masses by GVHD status and transplant type. For allogeneic transplant recipients, normalized median masses were averaged based on GVHD status for each time point. Lines were plotted using a third-order Bezier spline fit.

FIG. 6. Normalized median masses by GVHD status and transplant type. Each data point represents the median CD3+ T cell mass for one patient at the specified time point. Roman numerals indicate maximum acute GVHD grade, if present. Maximum severity of chronic GVHD is expressed as either mild, moderate, or severe, if present. Points with a black center indicate patient is now deceased.

FIGS. 7A and B. Estimated probabilities of acute GVHD occurrence at Lag 30 and 60 days. Lines represent different normalized median T cell mass values. Lag represents the time window for developing acute GVHD after a specific time point. For example, at Lag 30 (A) and a normalized median mass of 1.8 at (B) day 60, a patient would have an ˜80% probability of developing aGVHD within 30 days.

FIG. 8A-C. Schematic of HSLCI Vemurafenib dose-response assay. A, LCI set-up; B, Six patient-derived melanoma lines were plated in four wells each of a 24-well glass-bottomed plate (Cat #, Company, City) and incubated overnight under standard cell culture conditions (37° C., 5% CO₂). Following incubation, each cell line was dosed with either 0.1% DMSO or 1 uM, 5 uM, or 10 uM Vemurafenib and incubated for 24 hours. Finally, C, HSLCI was utilized to automatically and repeatedly measure cell masses for all treatment groups and cell lines over 10 hours. Briefly, a specialized camera (Phasics, SID4BIO, France) measured local phase shifts as light (670 nm wavelength) from a fiber-coupled LED passed through the cells, enabling the non-invasive quantification of dry cell mass. Phase images were collected at 20× magnification (Nikon Plan Fluor, Cat #, Japan) under standard cell culture conditions and processed using custom MATLAB programs designed to track individual cells between successive images and derive population growth rates for the characterization of kinetic response curves to Vemurafenib.

FIGS. 9A and B. Biomass accumulation response to Vemurafenib treatment. A) Normalized population median biomass versus time plots of each melanoma line exposed to either 5 uM Vemurafenib or 0.1% DMSO. Cells were synchronized prior to plating in glass bottom dishes. Each sample was imaged for three hours, after which either 0.1% DMSO (vehicle control) or 5 uM Vemurafenib (treatment) was administered. After dosing, plates were imaged for 23-27 hours using the HSLCI under standard cell culture conditions. Typical time between repeated measurements of the same location was 10-15 minutes. Each graph contains pooled data from 4 replicates. Error bars are ±SEM. B) All six synchronized cell lines were plated into a single 24-well glass bottom plate and dosed with either 0.1% DMSO, or 1 uM, 5 uM, or 10 uM Vemurafenib. After 24 hours incubation, the plate was imaged continuously for 10 hours with the HSLCI. Hourly growth rates were automatically calculated for individual cells in each sample by linear fit to the biomass versus time data. Data is from a single representative experiment (n=3). Boxplot notches are 95% confidence intervals for the indicated medians. Each dot overlaid on a boxplot represents the hourly growth rate of an individual cell.

FIGS. 10A and B. FDA-approved kinase inhibitor effect on M249R4 cells by HSLCI. Plots of hourly growth rates for Vemurafenib-resistant M249R4 cells during exposure to escalating doses of FDA-approved kinase inhibitors. After 24 hours inhibitor incubation, the plate was imaged by HSLCI continuously for 10 hours. Hourly growth rates were automatically calculated for individual cells in each sample by linear fit to the biomass versus time data. A) Seven-inhibitor screening panel. B) Three selected inhibitors with Vemurafenib and DMSO controls tested at finer doses. Data is from a single representative experiment (n=3). Boxplot notches are 95% confidence intervals for the indicated medians. Each dot overlaid on a boxplot represents the hourly growth rate of an individual cell.

FIG. 11. High level schematic representation of a high speed LCI (HSLCI) system.

FIG. 12. Relative Objective Position for Typical Scan The objective position in microns, showing the topography of a typical 24 well plate. Only the measurements of the forward scan are seen. This illustrates the need for an auto focus system if continuous scanning is desired. To start each run, the objective is placed near the bottom of its 100 micron range to attempt keeping any fluctuations in the plate surface within the range of the piezo.

FIG. 13. Ray Diagram of Laser Focus System A general arrangement of components for an optical beam deflection measurement can be seen. ƒ is the desired distance from the objective to the water/glass interface. As the plate is moved across the objective, any change in this distance, denoted by Δƒ, is observed at the QPD.

FIG. 14. Objective Ramp vs. QPD The semi-linear range in the middle of the graph is used as the dynamic range of the feedback input. The system typically stays within +/−0.5 μm of the center.

FIG. 15. Histograms of Error For each magnification, a series of 20 loops down one column of wells was performed. The density of QPD displacement signals from optimal focus is plotted. The dashed lines show the standard deviation calculated for the given data set.

### DETAILED DESCRIPTION

Live cell interferometry techniques allow the user to measure the phase shift of incident light through cells, which directly correlates to the cells dry mass. This label free technique is non-invasive and accurate enough to give detailed reports on the growth of the cells during multiple cell cycles over multiple days. This is useful because dry mass can be a direct result of biosynthetic and degradative processes within a cell, giving a precise metric of cell size during a response to drug treatments, cell death, etc. This technique gives a resolution much smaller than individual components of the cell, so the distribution of mass throughout the cell can be rendered as a function of time.

Methods for using LCI to determine the status of patients with respect to a risk for the occurrence of GVHD and for drug resistance during the treatment of cancers such as melanoma are provided. LCI is an automated, high-precision (<1% coefficient of variation) light microscopy technique capable of measuring the masses of thousands of individual living cells with picogram (pg) sensitivity. LCI does not require the fixation or staining of target cells, and is capable of tracking cells for up to four days.

The methods disclosed herein involve the real time assessment of cells of patients using LCI, in particular the assessment of cells that exhibit a change in response to incipient GVHD in transplant patients, or drug resistance in cancer patients. For both diseases, the methods may be employed before and/or during and/or after administration of a course of drugs (e.g. an immunosuppressive protocol for transplant patients and a melanoma treatment regimen for melanoma patients). Information acquired as a result of the LCI assessment includes the prediction of the development of GVHD or drug resistance, or the lack thereof, and is used to guide medical practitioners with respect to how to proceed with future treatments, e.g. whether or not to continue with or modulate administration of a particular drug, whether or not to switch to a different dosage of the same drug or to a different drug altogether, etc. These aspects are described in detail below for each of GVHD and cancer.

In addition, other aspects of the invention include novel device/apparatuses for performing high speed LCI (HSLCI) and methods of using the devices to assess cells as described above. In these aspects, the LCI equipment is modified to better suit a particular application, e.g. to better suit the identification and properties (and changes in properties) of one or more particular cell types over time.

The terms “subject” and “patient” are used interchangeably herein, and refer to an animal such as a mammal, which is afflicted with or suspected of having, at risk of, or being pre-disposed to GVHD or cancer. In general, the terms refer to a human.

### GVHD

Two types of GVHD are clinically recognized, acute and chronic. The methods of the present invention are used to predict the risk of and prophylactically treat both types. The acute form of the disease usually develops within the first three months after transplantation. The skin, liver and gastrointestinal tracts are the main targets of acute GVHD. Newly transplanted donor lymphocytes react to the host tissue antigens, resulting in cell damage to a variety of organs. The incidence rate of acute GVHD is estimated at 30-50% among patients receiving transplant from HLA-identical sibling donors, and 50-70% in patients receiving HLA-matched unrelated transplants. Severe acute GVHD (grade III-IV) occurs in up to 20% of recipients of related donors (Champlin, Blood 2000; 95:3702-3709) and up to 35% of unrelated donors (Castro-Malaspina, Blood 2002; 99:1943-1951, McGlave, Blood 2000; 95:2219-2225, Jagasia, Blood 2012; 119:296-307). Non-relapse mortality in patients who develop acute GVHD has been estimated to be in the range of 28% to 92%. Long term survival after grade I acute GVHD is greater than 90%, contrasting with 80%, 30%, and 10% for grades II, III, and IV, respectively. Chronic GVHD occurs in up to 60% of patients receiving HLA-identical sibling marrow grafts and 70% of patients receiving alternative donor marrow grafts who survive beyond day 100. (Lee, BBMT 2003; 9: 215-233). Symptoms of chronic GVHD usually present between 3 months and 2 years after allogeneic transplantation, about two thirds develop within the first 12 months. Manifestations of chronic GVHD may be restricted to single organ or tissue, but typically 2 or 3 organs are involved. The organs most commonly affected are the skin, mouth, and eyes, with more than 50% of patients demonstrating these manifestations. Other disease sites include the liver, lungs, gastrointestinal tract, musculoskeletal system, and female genital organs.

SCT patients are routinely given one or more types of immunosuppressive treatment before and after transplant to prevent rejection and also to prevent GVHD. Typical treatments include the administration of low doses of the chemotherapy drug methotrexate, along with the drug cyclosporin (which is typically continued for about 4-6 months after the transplant), or a combination of mycophenolate mofetil (MMF) and tacrolimus. These medications are tapered off after 4-6 months in order to enable a graft vs. malignancy effect.

However, routine GVHD prophylaxis puts patients at risk for opportunistic infections as well as malignancy relapse, while withdrawal of immunosuppression is frequently associated with the development of clinically significant GVHD. Without the guided withdrawal of immunosuppression, rates of morbidity and mortality in allogeneic transplant recipients remain unacceptable. As the primary effectors of GVHD, T cells and their subsets provide a solution as a unique target for the mass-based assessment of post-transplant alloreactivity. In some aspects, the present disclosure describes methods and techniques for the comprehensive assessment of the post-transplant activation of donor-derived T cells in subjects who have undergone a stem cell transplant (SCT), in order to predict or detect the occurrence of acute or chronic GVHD.

The antigen-driven proliferation of donor T cells in patients who develop GVHD manifests itself as an increase in the size and mass of individual T cells as they become activated following antigen exposure and second signaling. In contrast, homeostatic proliferation results in slower growth, primarily preserving T cell clones without significant expansion and precluding the development of GVHD. As such, patients responding to a library of alloreactive antigens exhibit a different post-transplant T lymphocyte mass spectrum than patients who have a more generalized polyclonal recovery pattern (with T cell clones responding to pathogen targets as well as tumor targets). Further, the mass spectrum of patients undergoing allogeneic SCT is significantly different from those undergoing autologous SCT because of a large library of recipient antigens that the non-tolerized donor T cells encounters in an allograft.

Using the LCI assessment techniques described herein, patients at risk of or in the process of developing GVHD can be identified. Thus, if, after a transplant, a patient is, for example, being gradually weaned from prophylactic immunosuppressive therapy but an increased T cell mass concerning for the onset of GVHD is detected, the attending physician can reinstitute administration of one or more immunosuppressants, which may be the same or different from that which was initially used. Alternatively, if GVHD is detected in a patient that is on an immunosuppressive therapy, the physician can increase the dose of the therapeutic agent being used, or change to a different agent, or add one or more additional immunosuppressive treatments to the therapeutic regimen. Similarly, immunosuppressive therapy may be started again in a patient who has stopped therapy if GVHD is detected. The methods described herein may be used to monitor transplant subjects before transplant (e.g. donor stem cell product infused into the recipient to establish a base line and/or to detect the initial effects of pre-procedure immunosuppression), and after transplant during the initial recovery period, as well as thereafter, to detect and/or rule out the onset of GVHD. In some aspects, the patients are monitored for at least about 100 days or longer (e.g. for 1-12 months, or indefinitely on a monthly or yearly basis, etc.) In particular, in some aspects, patients are monitored at least at 30 and 100 days, e.g. at 10, 20, 30, 40, 50, 60, 70, 80, 90 and 100 days. For GVHD patients, it has been found that it is especially helpful to obtain measurements at at least about 30 and 100 days post-transplant, as the mass of T cells in patients with or at risk of GVHD tends to peak at about 30 days, then decrease and then peak again at about 100 days.

Typically, the onset of GVHD is characterized by an increase or gain in the median T-cell population mass of at least about 1-20%, e.g. at least about 1%, 5%, 10%, 15%, or 20% compared to, e.g. the donor T cell mass prior to transplant or the T cell mass of the same patient at T=0, where T=0 is a T cell mass measurement obtained for the transplant patient within e.g. 24 hours of the transplant. Alternatively, a large data set could be collected to determine the range of ‘normal’ T-cell mass distributions, as a function of age, race, time before or after transplantation, medical condition, specifically GVHD and infections, etc. A patient T cell mass measurement would then be compared to the appropriate matching values for their demographic or condition. Serially measured T cell masses following stem cell transplantation may then be used as a biomarker to monitor for GVHD risk in real time. This will allow early diagnosis of GVHD, as well as guide the intensity of immunosuppressive therapy. For example in a patient with a rising average T cell mass, in the absence of signs and symptoms of infection, GVHD will be the most likely cause of rising T cell mass. The T cell mass measurements will in this case serve to guide the schedule of immunosuppressive therapy, whether it should be intensified or tapered as time passes following transplant. In patients who develop chronic GVHD following cessation of immunosuppression, T cell mass again may be valuable in helping distinguish this from other disorders.

Alternatively, or in addition, the measured T cell mass may be compared to one or more other reference values (which may be positive or negative) obtained from the transplant donor at the time of collection (who is not at risk for GVHD), other transplant recipients with GVHD, other transplant recipients who do not have GVHD, and/or healthy control subjects who have not undergone a transplant. A subject who is identified as having GVHD may or may not have any other symptoms of GVHD; preferably, the methods described herein detect the onset (or recurrence) of GVHD before other symptoms are present.

Should GVHD be indicated, the initial immunosuppressive treatment may be continued, or restarted, and/or other treatment options may be used. For example, exemplary alternative drugs include but are not limited to: tacrolimus or sirolimus (Rapamune) or other mTOR inhibitors, various steroids such as prednisolone and methylprednisolone, steroid creams to reduce skin GVHD, steroid eye drops for GVHD affecting the eye, etc. Other treatment options include but are not limited to: extracorporeal photopheresis (ECP), monoclonal antibodies such as rituximab, tyrosine kinase inhibitors (TKIs) such as imatinib, etanercept, mycophenolate mofetil (MMF), interleukin 2 and pentostatin.

One or more of these agents or measures may be started to prevent or treat the symptoms of GVHD, and/or the dosage and/or frequency of administration may be increased until symptoms subside. Exemplary drugs used to prevent GVHD include alemtuzumab (Campath) and antithymocytic globulin (ATG).

Alternatively, if a patient is being gradually withdrawn from immunosuppressant therapy, the methods described herein can monitor the status of T cell mass in order to confirm that the withdrawal is progressing successfully and can be continued, or even hastened, if no evidence of GVHD is detected. Aside from the potential use on stem cell transplant recipients, LCI may be similarly applied in solid organ transplant (SOT) recipients. A rising T cell mass in a SOT recipient may herald graft rejection and the need to increase the dose of immunosuppressants. Alternatively, a near base line (pre-transplant recipient) T cell mass may imply tolerance of the donor solid organ and that immunosuppression levels may be safely reduced to minimize opportunistic infection and malignancy risk.

T cell mass studies as described herein may also be used to assess the effectiveness of immunotherapy treatments of cancer. Because T cell activation is associated with a higher T cell mass, the application of immunotherapy may result in the average T cell mass increasing in patients who are responding to the treatment, as opposed to the patients who do not respond. This is an important distinction to make, given the side effect profile of these agents. Additional applications include monitoring disease activity in auto-immune disorders such as scleroderma and Crohn's disease, which may also have increased average T cell mass as a consequence of auto reactivity.

### Cancers

### Melanoma

About 50% of melanomas have a mutated or activated BRAF gene which has provided an important new direction in the treatment of melanoma. There are 3 drugs that inhibit BRAF. Dabrafenib (Tafinlar), vemurafenib (Zelboraf) and trametinib (Mekinist) have been FDA approved e.g. for people with both stage IV and stage III melanoma that cannot be surgically removed. These drugs, taken as a pill, are at present specifically used when the melanoma tumors have a V600E or V600K mutation in the BRAF gene. All three drugs, and combinations thereof such as dabrafenib and trametinib, are approved for use in patients with locally advanced stage III melanoma that cannot be removed by surgery and for patients with stage IV melanoma, if the melanoma has the mutated BRAF gene. The drugs shrink the tumors in the majority of patients, and can extend patients' survival by up to about one year. However, unfortunately all patents become resistant to whatever drug is used within about one year or less.

It is important to accurately identify if and when a patient has become resistant to one of the drugs or a combination of the drugs. Conventional tests for tumor cell drug resistance depend on the isolation and cultivation of tumor cells in vitro. However, such cultures are subject to in vitro clonal selection, which can result in false positive results. In addition, patient melanoma samples can grow, survive, and respond to drugs for only 1-2 days in culture following isolation. Thus, in order to efficaciously predict suitable drug choice(s) for patients being treated for melanoma, speed and accuracy are essential to avoid artifacts of in vitro clonal selection of resistant subclones and to be effective within the available time window. LCI biomass profiling, in particular high speed LCI, fits well within these significant constraints to bring a rational, systematic drug prediction and selection approach into clinical use. LCI provides a solution via the automated measurement of tumor cell biomass.

Patients with solid organ cancers, such as melanoma, may have LCI technology applied at different levels. First the LCI assay can be used at the time of diagnosis to determine which drug will be most effective in disrupting tumor growth. At the time of relapse, similar in vitro assays help in drug selection for salvage therapy. In patients undergoing immunotherapy, T cell mass can be monitored by LCI to assess treatment response.

LCI or HSLCI allows for the detection and measurement of drug resistance at a much earlier timepoint as compared to previous methods. Using these methods, resistant cells within a primary tumor sample or circulating resistant cells obtained from a blood sample can be visualized before a patient shows clinical signs of resistance.

Any biological sample suspected of containing resistant cancer cells as described herein may be tested according to methods of the present invention. By way of non-limiting examples, the sample may be tissue (e.g., a tumor biopsy sample), urine, sputum, blood, or a fraction thereof (e.g., plasma or serum). Methods of detecting cancer cells by setting culture conditions in which only cancer cells grow are known in the art.

The term “tumor sample” means any tissue tumor sample derived from the patient. Said tissue sample is obtained for the purpose of the in vitro evaluation. The sample can be fresh, frozen, fixed (e.g., formalin fixed), or embedded (e.g., paraffin embedded). In a particular embodiment the tumor sample may result from the tumor resected from the patient. In another embodiment, the tumor sample may result from a biopsy performed in the primary tumor of the patient or performed in a metastatic sample distant from the primary tumor of the patient.

Using LCI, or HSLCI, a clinician can prescribe and administer a first drug or a first combination of drugs, such as one BRAF inhibitor or a combination of two BRAF inhibitors, and, upon early detection of the development of resistance to the first drug, can discontinue its use immediately and prescribe and administer another drug, or drug combination, or other treatment modality, to which the patient is not resistant. Accordingly, in this aspect of the disclosure, a patient is diagnosed with a melanoma suitable for treatment e.g. with one or more BRAF inhibitors for a period of time, e.g. several weeks, or months, or even years. During the treatment period, biological samples are taken from the patient e.g. once per week, once every few weeks, once per month, etc. and LCI is used to determine whether or not resistance to the drug or combination of drugs has developed. If resistance has not developed, the treatment can be continued. If resistance has developed or is developing, treatment with the first drug or drug combination ceases and an alternative treatment is pursued.

The methods of the invention can also be used to detect relapse of a cancer at an early timepoint. A clinician may obtain periodic samples from a patient (e.g. a liquid biopsy) to find cells of unusual mass, shape or growth rate, indicating relapse. Alternatively, patient T cell mass could be periodically screened, searching for elevated mass indicative of an anti-tumor immune response, to detect relapse.

Typically, drug resistance is characterized by detecting, ex vivo, the presence, in a biological sample from a subject, of cancer cells that are resistant to the drug with which the patient is being treated. Cells in the sample are tested in the presence and absence of the drug with which the patient is being treated, and the growth rate of the cells is measured. An increase in survival of cells, as indicated by an increase in growth rate is detected by measuring the mass of cells in the sample, since growth correlates positively with an increase in cell mass. For example, an increase of about 1-20%, e.g. at least about 1%. 5%, 10%, 15%, or 20%, compared to one or more reference values, indicates that tumor cells of the patient are becoming or have become resistant. The one or more reference values (which may be positive or negative) include but are not limited to: a reference value obtained from the subject prior to the onset of resistance, a reference value obtained by measuring the growth rate of cancer cells from other melanoma patients treated with the same drug but whose cells were not resistant, or by measuring the growth rate of cancer cells from other melanoma patients treated with the same drug whose cells had become resistant, or by measuring the growth rate of suitable reference cells from healthy subjects who do not have melanoma. If drug resistance is indicated, another different treatment option is pursued.

Alternative treatments that are pursued include but are not limited to, for example, administration of another kinase inhibitor that targets a different growth pathway, or a combination of different agents, in an attempt to overcome the resistance. A large number of kinase inhibitors are available, and many options for combinations of those, but unfortunately the oncologist has no rapid, reliable way of knowing which of the possible options is most likely to work in a particular patient. However, LCI or HSLCI can be used to assist the selection.

Thus, an aspect of the invention also relates to a method for screening a plurality of candidate drugs or combination of drugs useful for treating cancer (e.g. melanoma) that is resistant to one or more previously administered drugs. In some embodiments, the method includes performing an LCI or HSLCI analysis of a biological sample having resistant cancer cells obtained from the patient to obtain a plurality of dose response curves for each candidate drug or combination of drugs in the group of candidate drugs or combination of drugs,

wherein the plurality of dose response curves is obtained at
i) a highest concentration of each candidate drug or combination of drugs equal to a peak serum concentration tolerated by patients, and
ii) one or more lower concentrations of each candidate drug or combination of drugs, each of which is lower than the highest concentration.
   The drug or combination of drugs that inhibits growth of the resistant cancer cells at the lowest of the one or more lower concentrations that are tested is then selected and administered.

In some aspects, a kinase inhibitor different from the first one (or first combination) is the second treatment and LCI is employed to select one or more suitable kinase inhibitors to use for the second treatment. In this aspect, a sample of resistant melanoma cells is obtained from ti the patient and an LCI analysis is conducted to obtain a dose response curve for each drug in a panel of candidate FDA approved drugs, in order to determine which agents in the panel work for the particular BRAF-inhibitor resistant melanoma cells in the sample. The cells in the sample are exposed to e.g. i) the highest test concentration of each agent equal to the peak serum concentration tolerated by patients, as given by the published clinical trials data; and ii) a series of increasing lower concentrations, in order to identify one or more inhibitors that slow or arrest growth of the melanoma cells. The agent that works at the lowest concentration vs the peak (highest) concentration is the optimum choice. This type of analysis is described in Example 2.

### Other Cancers

The methods of the invention are generally applicable to all cancer types, as this disease involves dysregulation of cell growth. Thus, in cancer, cell biomass properties and accumulation rates are altered. Most cancer therapeutic strategies involve inducing cell death, which is detectable via LCI or HSLCI as a loss of cellular biomass. Alternatively, some agents arrest growth, which is detectible as a reduction in biomass accumulation rate, or lack of biomass accumulation.

LCI or HSLCI is particularly well suited to therapy selection for solid tumor cancers, because it can measure cells and cell clusters, clumps and organoids with equal effectiveness. It is effective with minute amounts of material—few cells per sample at minimum—making testing of multiple agents practical from fine needle aspirate biopsies, cells extracted from urine and sputum, and even circulating tumor cells isolated from the blood.

We have tested with the HSLCI system melanoma, breast and brain cancer. Exemplary cancers to which the methods described herein are applicable include but are not limited to liquid discohesive tumors such as lymphoma and leukemia, brain, bladder, colon, endometrial, renal cancer, pancreas, thyroid, lung, liver, breast, and myeloma. In some aspects, the cancer is not myeloma or breast cancer.

### High-Speed LCI (HSLCI)

Live cell interferometry techniques allow the user to measure the phase shift of incident light through cells, which directly correlates to the cells dry mass. This label free technique is non-invasive and accurate enough to give detailed reports on the growth of the cells during multiple cell cycles over multiple days. This is useful because dry mass can be a direct result of biosynthetic and degradative processes within a cell, giving a precise metric of cell size during a response to, for example, drug treatments. Because this technique gives a resolution much smaller than individual components of the cell, the distribution of mass throughout the cell can be easily rendered as a function of time.

Previously, the dry mass of two cell samples with a 4×4 imaging grid of each sample were examined using a quadri-wave lateral shearing interferometer (QWLSI) with z-ramp based focus. The largest problem with the previous LCI setup was the time it took to insure the camera was in focus. With this earlier system, a contrast metric was used and a frame/stage position was found as best focus, a focusing process that took approximately eight seconds per image. Ideally, if minimal time lapse between frames is desired, the stage needs to run continuously. This requires that the objective remain at the desired distance away from the sample, even if the well shape/thickness/height changes, necessitating a feedback loop that monitors the distance between the objective and sample and makes any necessary changes to ensure that the distance remains constant.

It has been discovered that incorporation of an autonomous autofocus component into the LCI apparatus solves this problem. The autofocus component advantageously employs an optical beam deflection position measurement to overcome problems due to mechanical instability, changes in glass thickness of the sample container, thermal expansion, etc. This feature allows the sampling stage to move continuously while accurately acquiring e.g. four frames per second (fps), regardless of the topography of the sampling surface. Thus, the invention also provides new high speed LCI (HSLCI) apparatuses which include an autofocus component, as well as methods of using the apparatuses. Using HSLCI, it is possible to examine three times the sampling grid per sample in a fraction of the time, allowing 24-48 samples in parallel to be measured accurately and continuously for from 1-72 hours.

Fundamental components of the HSLCI include the following:

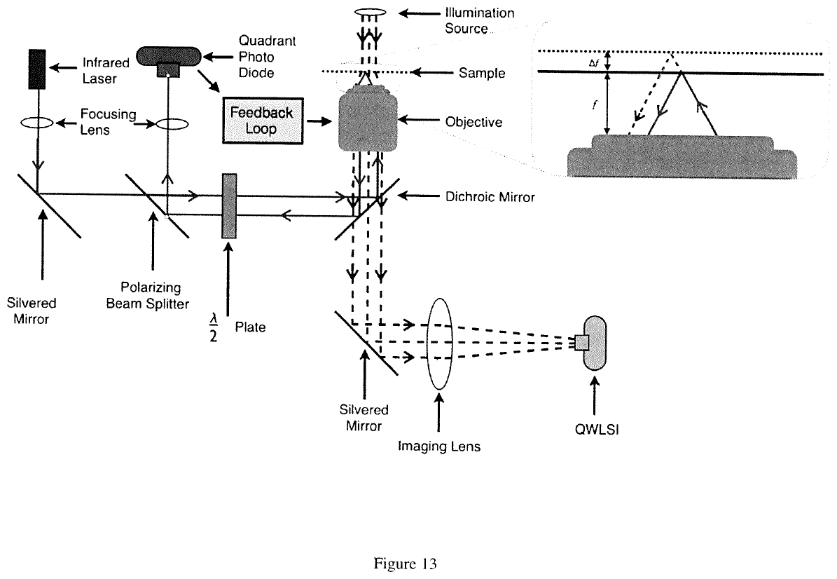

1. A camera with an objective. The objective is positionable, i.e. is capable of moving up and down along a y axis with respect to the sample that is interrogated. In particular embodiments, the camera is a phase contrast camera capable of acquiring images of light reflected from the sample from a source of illumination positioned behind the sample, as shown in FIG. 13.
2. The HSLCI includes an observation chamber for containing cells of interest. In some aspects, during data acquisition, the chamber is placed on a stage that is moveable in the x and y directions with respect to a y axis which is parallel to the direction of light transmission in the microscope objective field of view. The chamber preferably moves continuously during data acquisition to eliminate or lessen the effects of acceleration that can result from stopping and restarting movement. Generally, the chamber moves in a single (e.g. x) first direction in a pattern that permits acquisition, by the camera, of images of cells in a single “line” that is exposed to the objective field of view. At the end of the “line”, the chamber is offset slightly (e.g. in the y direction) and the chamber travels back in the opposite direction so that a second line is exposed, and so on until the entire chamber is scanned. The second (and subsequent lines) do not overlap the preceding line. Generally, the cells are moved across the field of view so that at least about 50 cells, and preferably more than about 100 cells, are detected per second, e.g. at least about 50, 60, 70, 80, 90, 100, 150, 200, 250, 300, 350, 400, 450, 500 or more cells are detected per second. In alternative aspects, the sample chamber itself does not move but cell movement occurs via a flow channel by which individual cells are circulated/moved across the field of view.

In some aspects, the cells that are imaged are present in a liquid medium, e.g. a suitable culture or maintenance medium. However, dry cells are also measurable if mass alone is the desired metric. Live cells are required for time-dependent physiologic measurements. Most cell types require that e.g. a 5% CO₂ environment and an elevated temperature (generally about 37C) be maintained during analysis for maximum viability. However, for a particular purpose, the environmental parameters may be adjusted as needed, e.g. the composition of the medium, the ambient atmosphere and/or the temperature may be altered. Samples may comprise single isolated cells (e.g. as small as about 1 micron), cell clusters (e.g. about 10-50 microns), or ‘organoids’ containing many cells of size up to e.g. about 100-150 microns, or even combinations of these. As used herein, an “organoid” refers to an artificially grown mass of cells or tissue that resembles an organ, i.e. that has at least one or more properties of an organ such as composition, function, etc. Cells may be attached to the chamber surface, suspended in solution or within solid transparent matrix material such as gelatin.

3. A quantitative phase detector for cell mass determination. The preferred metric is that the sample rate must be high, greater than 1 million pixels per second. Examples of types of quantitative phase detectors that may be used in the practice of the invention include but are not limited to:

Shack-Hartman wavefront sensor, (as described in Example 4 below); reference beam interferometers (e.g. Michelson, Mach-Zeder, Linnik, Fabry-Perot, etc.); holographic detectors; single beam heterodyne interferometers, etc.

4. The HSLCI devices disclosed herein require an autofocus component to continuously and automatically calculate, control and maintain a distance between the sample and the microscope objective that corresponds to the optimal operational depth of focus of the system. This component insures that the sample is perpetually within the requisite distance from the objective in order to achieve optimal focus and image clarity. The margin of tolerance for this distance depends on the objective numerical aperture or magnification and generally ranges from about 0.25 microns (for 40-60× magnification) to about 100 microns (for 1× magnification).

In some aspects, a coaxial optical beam deflection displacement sensor is employed as the autofocus component. Such a sensor is advantageous because it has positional sensitivity better than 0.5 microns, and high detection bandwidth (>1000 Hz), so the sample can be translated very rapidly past the objective. The coaxial beam system is well suited because the point of measurement is within the microscope field of view at all times. However, other positional sensors may also be used, if they satisfy the resolution and bandwidth requirements of the system.

Regardless of the distance detection scheme, a feedback loop (such as a microcontroller feedback loop) and a precision actuator are also included in the system. The feedback loop is configured so as to: receive an electrical signal corresponding to the position of the microscope objective; calculate whether or not the objective is positioned so that the distance between the objective and the sample are optimal, and if not, calculate the offset that is required to raise or lower the objective to attain the optimal distance; using this information, generate an offset signal; and transmit the offset signal to the precision actuator.

The precision actuator then causes the objective to move as dictated by the offset signal, and the proper distance is reestablished. In some aspects, a piezo actuator is used to move the microscope objective because it has <0.5 micron precision and a high bandwidth (>100 Hz).

This mechanism operates continuously during data acquisition and eliminates the need of previous LCI systems to pause and refocus repeatedly. Thus, the present HSLCI systems acquire data much more rapidly and can be used for a wider variety of applications.

While in most aspects, the microscope objective moves in relation to the observation chamber, this is not always the case. Systems can be configured so that the chamber also moves in a y direction to maintain the correct distance, or so that both the microscope objective and the chamber move to do so. Given the size, weight, etc. of these respective components, typically it is the camera objective that is displaced to maintain the desired distance.

In addition, the autofocus component comprises a light source, typically an LED. This light source generates light that travels, via a pathway comprising e.g. at least one focusing lens, at least one reflective mirror, and a polarizing beam splitter which sends a reference portion of the light to the feedback loop and a portion of the light to the camera objective and to the glass-water interface of the observation chamber. The light is then reflected back from the interface, through the objective and to the feedback loop. Any change in distance between the objective and surface gives a change in distance from the beam going out of the objective to the beam re-entering the objective. This signal is then transmitted to a sensor where the intensity/position of the laser is converted to a dynamic analog voltage signal. Once this signal is conditioned, it serves as the input to the feedback loop.

For wide-field detectors, a pulsed light source to create ‘strobed’ illumination is used, which prevents blurring of rapidly moving objects in the field of view. The strobe needs to be intense enough to provide illumination over a duration of 1 millisecond or less, down to microseconds. Typically, this could be an electronically gated LED, or an electronically modulated laser, or a flash lamp. In most cases ‘spatially coherent’ light emanating from a single point source, such as the end of an optical fiber, is required. ‘Temporally coherent’ light, such as a laser is not required in many configurations, but can be used, and is in fact needed for holographic detection schemes. Scanned single beam interrogation methods, such as heterodyne interferometers, need to be scanned at rates of greater than 1000 pixels per second.

In addition, the autofocus component comprises an electronic system to coordinate the light from illumination source with the motion of the stage on which the observation chamber is placed. For example, if the light is intermittent (e.g. strobed, flashed, etc.) it is necessary to coordinate the flashes of light, the opening and closing of the camera shutter and the position of the sample that is in the field of view to achieve maximal imaging results. In some aspects, this is accomplished using a dedicated microprocessor that sends ‘triggers’ to synchronize image acquisition with strobing.

5. The HSLCI devices disclosed herein also comprise one or more computer processors comprising image analysis software with the following functions: quantitative phase calculation from the raw image or data stream, post processing to remove artefacts that interfere with measurement of cells in the image, and an implement an algorithm to identify cells within the image and calculate their mass. Finally, in many cases mass changes of one object over time are of interest, so an additional step which identifies the same object in two images collected at different times is required. This last step is referred to as “cell tracking”. To obtain the necessary throughout, this image analysis pipeline requires the use of one or more of a multi-core central processing unit (CPU) having two or more processing units on the same integrated circuit, a graphics processing unit or a field-programmable gate array (FPGA) unit.

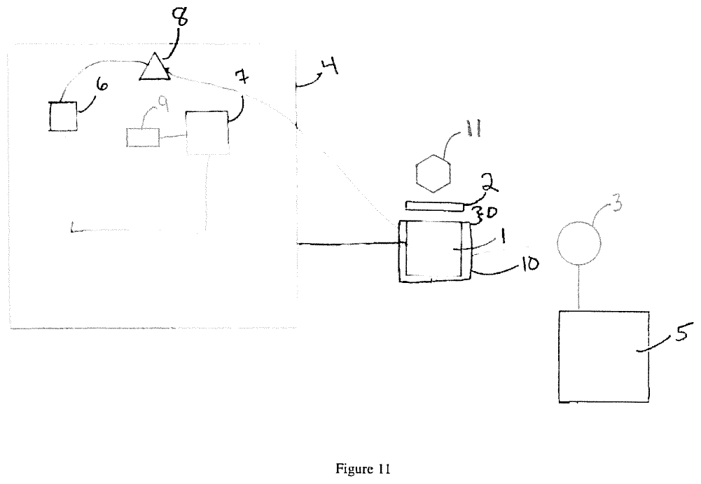

FIG. 11 shows a schematic representation of an HSLCI system as disclosed herein. In the figure, camera 10 with camera objective 1 is shown positioned so as to obtain images from cell samples disposed within observation chamber 2. Camera objective 1 is operatively linked to quantitative phase detector 3. Quantitative phase detector 3 computes cell mass determination based on phase contrast images obtained from a camera which include camera objective 1. Autofocus component 4 is operably linked to and ultimately controls the position of camera objective 1 with respect to sample chamber 2, i.e. the distance D between camera objective 1 and observation chamber 2 via the individual components disposed therein. Autofocus component 4 comprises light source 6 and feedback loop 7, both of which are described in detail elsewhere herein. Electronic system 8 controls coordination of light emitted by light source 6 and the movement of camera objective 1. Processor 5 is operably linked to receive input at least from quantitative phase detector 3. Using input from quantitative phase detector 3, processor 5 determines the identity of cells within the sample and calculates their mass. This information is output in a form that is useful to the user, e.g. as an image on a computer screen, a printed or printable image or list, etc.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting.

Where a range of values is provided, it is understood that each intervening value between the upper and lower limit of that range (to a tenth of the unit of the lower limit) is included in the range and encompassed within the invention, unless the context or description clearly dictates otherwise. In addition, smaller ranges between any two values in the range are encompassed, unless the context or description clearly indicates otherwise.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### EXAMPLES

### Example 1. Reconstituting Donor T Cell Mass as a Biomarker for Alloreactivity Following Stem Cell Transplantation

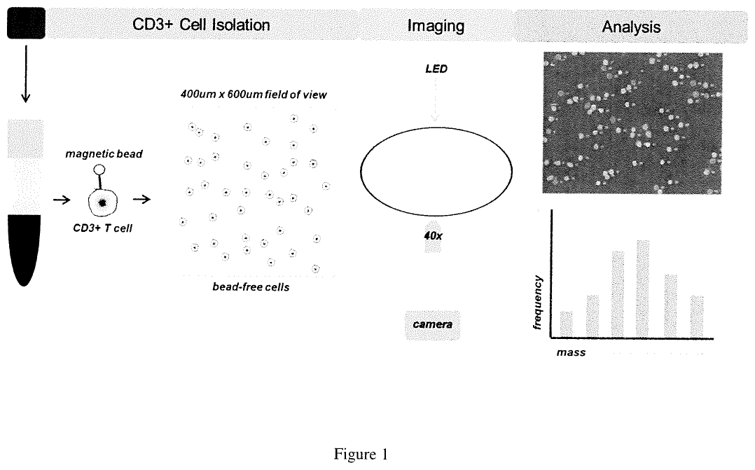

This example describes studies of the response of donor T cells upon in vivo post-transplant exposure to recipient antigens. Specifically, LCI was utilized to non-invasively measure the masses of thousands of individual live CD3+ T cells isolated from donor apheresis products and the whole blood of patients undergoing autologous and allogeneic SCT. Mass spectra were generated from LCI data for each donor apheresis product sample and transplant recipient samples at regular intervals during the first 100 days post-transplant to identify signatures of acute and chronic GVHD prior to the onset of clinical symptoms (FIG. 1).

### Patients, Materials, and Methods

Consecutive patients undergoing myeloablative conditioning and stem cell transplantation between May 2015 and May 2016 were enrolled on a prospective study approved by Virginia Commonwealth University's Institutional Review Board (VCU-IRB #HM20004916). In this study, donor apheresis product and recipient blood samples were collected for T cell mass estimation. Allogeneic SCT recipients received grafts from HLA-A, B, C and DRB1 matched related (MRD, n=3) and unrelated donors (MUD, n=11); four patients underwent autologous SCT (Table 1). CD34+ and CD3+ cell counts in the infused product, time to myeloid engraftment, lymphocyte recovery, and donor derived T cell count following transplantation were also determined.

### Hematopoietic Stem Cell Transplantation

Patients conditioned with myeloablative regimens were included in this study; conditioning regimens are detailed in Table 1. All patients undergoing allogeneic SCT received rabbit anti-thymocyte globulin (Thymoglobulin) either 3.5 mg/kg (MRD) or 5 mg/kg (MUD) starting from day −3 to day −1. GVHD prophylaxis was with either tacrolimus or cyclosporine in combination with methotrexate (MTX) or mycophenolate mofetil (MMF). Immunosuppression was tapered after day 100 in most patients. Antimicrobial and antifungal prophylaxis was administered. Routine surveillance for cytomegalovirus (CMV) and Epstein Barr virus (EBV) was carried out using PCR. Patients had blood and T cell chimerism measured, as well as absolute CD3+ cell counts, at one, two, and three months following SCT.

TABLE 1
Patient Demographics.

| | n = 18 | |
|---|---|---|
| Median Age, y (range) | 49 | (24-65) |
| Gender | | |
| Female | 9 | |
| Male | 9 | |
| Disease | | |
| Acute lymphoblastic leukemia | 3 | |
| Hodgkin's lymphoma | 2 | |
| Myelodysplastic syndromes | 4 | |
| Chronic myelogenous leukemia | 1 | |
| Non-Hodgkin's lymphoma | 3 | |
| Acute myeloid leukemia | 2 | |
| Multiple myeloma | 1 | |
| Myelofibrosis | 2 | |
| Donor Type | | |
| Matched unrelated | 11 | |
| Matched related | 3 | |
| Autologous | 4 | |
| Conditioning Regimen* | | |
| TBI/Cyclophosphamide (TBI/Cy) | 2 | |
| Busulfan/Cyclophosphamide (Bu/Cy) | 6 | |
| Busulfan/Fludarabine (Bu/Flu) | 4 | |
| Fludarabine/Melphalan (Flu/Mel) | 2 | |
| BEAM | 3 | |
| Melphalan (Mel) | 1 | |
| CMV Status (Donor/Recipient) | | |
| +/+ | 7 | |
| +/− | 3 | |
| −/+ | 4 | |
| −/− | 0 | |
| GVHD Prophylaxis | | |
| Tacrolimus-Methotrexate | 6 | |
| Tacrolimus-Mycophenolate mofetil | 4 | |
| Cyclosporin A-Methotrexate | 2 | |
| Cyclosporin A-Mycophenolate mofetil | 1 | |
| Median CD34 Dose (E6 cells/kg) (range) | 4.59 | (3.11-9.40) |

*Scheduling and dosing for conditioning regimens were as follows
TBI/Cy 2 Gy TBI in 6 fractions bid days −6, −5 −4, Cyclophosphamide 60 mg/kg/day days −3, −2
Bu/Cy Busulfan 0.8 mg/kg for 16 doses days −7, −6, −5, −4, −3; Cyclophosphamide 60 mg/kg/day days −3 −2
Bu/Flu Fludarabine 40 mg/m² days −5, −4, −3, −2, Busulfan 130 mg/m² days −4, −3, −2
Flu/Mel Fludarabine 30 mg/m² days −6, −5, −4, −3; Melphalan 140 mg/m² day −2
BEAM Carmustine 300 mg/m² day −7; ara-C 100 mg/m²) for 8 doses days −6, −5, −4, −3; Etoposide 100 mg/m² for 8 doses days −6, −5, −4, −3, Melphalan 140 mg/m² day −2
Melphalan 200 mg/m² day −2

### GVHD Diagnosis and Classification

Clinical outcomes were determined from the day of transplantation. Acute and chronic GVHD, as well as significant infection events, were documented, including bacteremia, CMV and EBV viremia. Glucksberg criteria were used to grade acute GVHD and NIH consensus criteria for chronic GVHD.

### Sample Acquisition and CD3+ T Cell Isolation

Donor samples were obtained from apheresis products, with a 2 mL aliquot provided for each patient. Transplant recipient whole-blood samples (3 mL) were drawn using EDTA-coated vacuutainer tubes at 14±3, 28±3, 56±3, and 100±3 days post-transplant. After acquisition, whole-blood samples were stored at +4 degrees Celsius (° C.) for no longer than four hours prior to cell isolation.

CD3+ T cells were isolated directly from donor products and patient whole blood samples using the Dynabeads FlowComp Human CD3 Kit (ThermoFisher Scientific, Cat #11365D, Waltham, Mass.). Isolated cells were resuspended in 500 μl sterile phosphate-buffered saline (PBS) (ThermoFisher Scientific, Cat #14040133).

### Live-Cell Interferometric Imaging

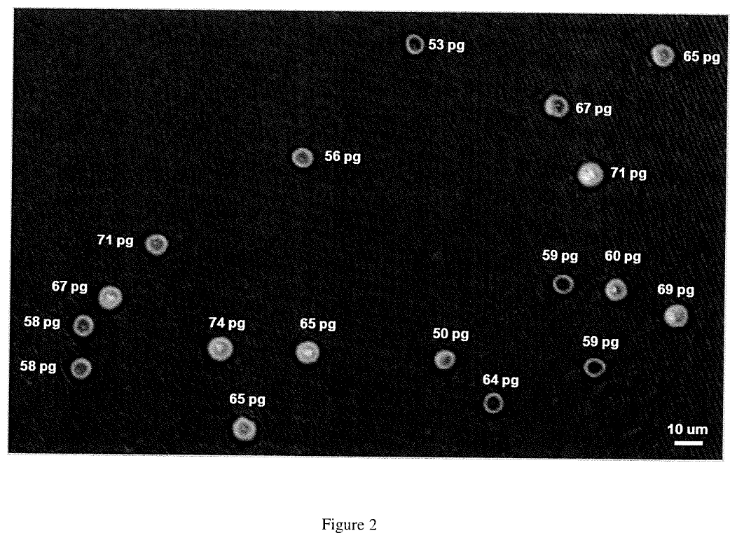

Isolated CD3+ cells were transferred to optical glass-bottomed cell culture dishes (WPI, Cat #FD35-100, Sarasota, Fla.) to a maximum concentration of 1×10⁶ cells/mL in a 3 mL volume of sterile PBS. Dishes were allowed to reach thermal equilibrium (37° C.) for 30 minutes in the LCI microscope chamber prior to data collection. Individual CD3+ cell mass measurements were obtained using the LCI. Briefly, a specialized camera (Phasics, SID4BIO, France) measures local phase shifts as light (670 nm wavelength) from a fiber-coupled LED passes through living cells, enabling the non-invasive quantification of dry cell mass. For all cells in each sample, 20 phase and standard brightfield images were collected at 40× magnification (Nikon Plan Fluor, NA 0.75, #MRH00401, Japan) under standard cell culture conditions (37° C., 5% CO₂) using phase (Phasics SID4BIO, France) and color (Basler ac645, USA) cameras, respectively (FIG. 2).

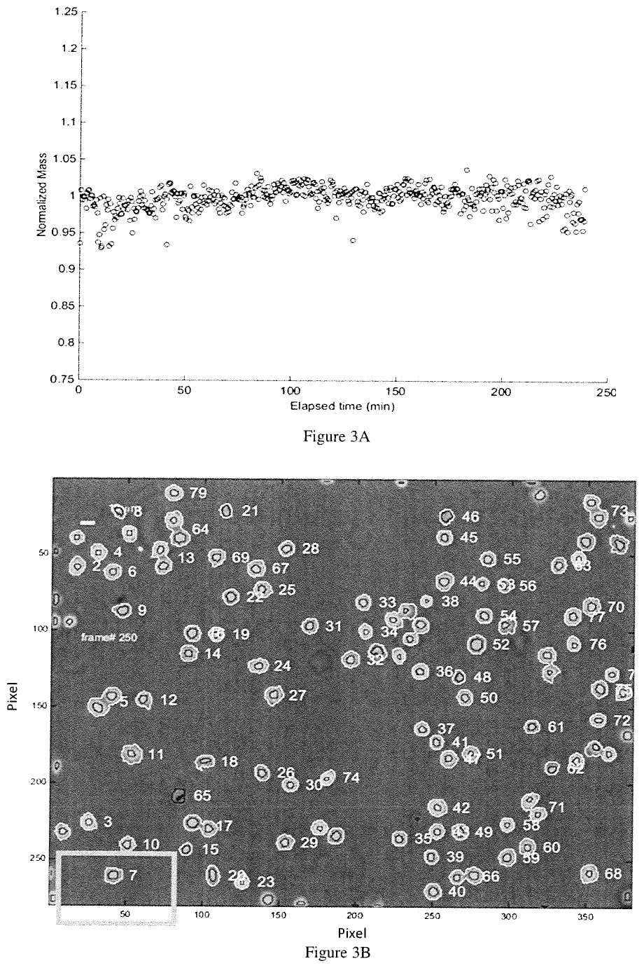

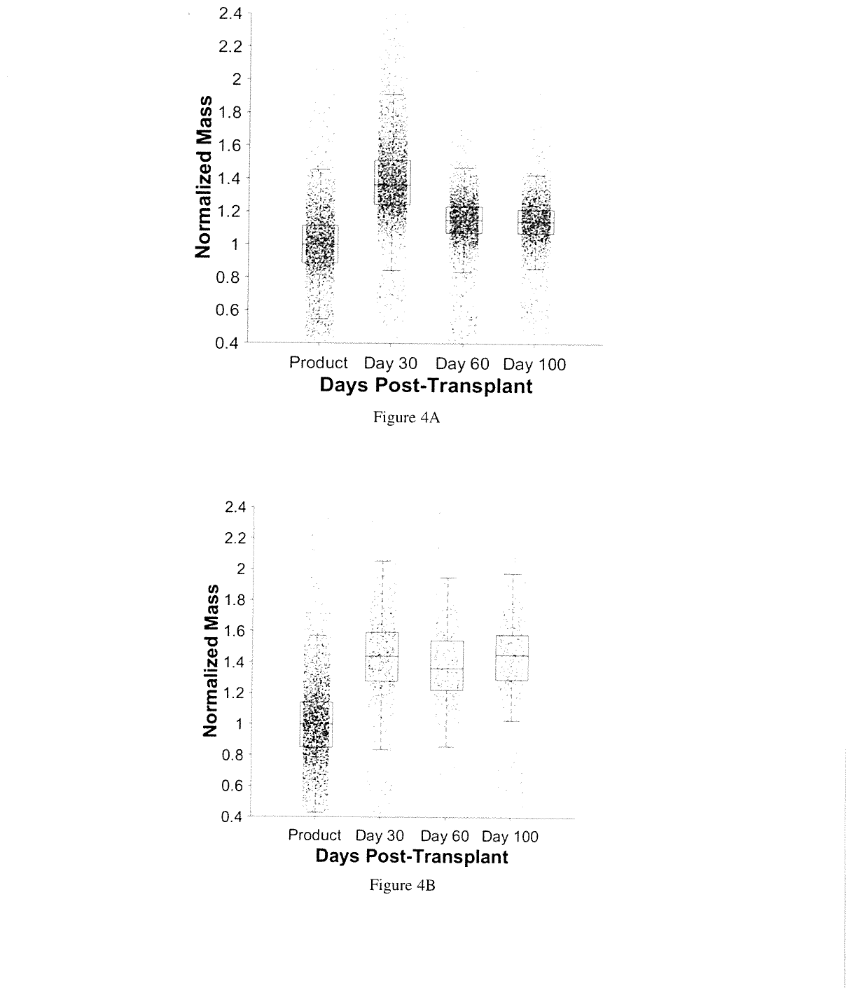

Initial validation testing revealed that, when imaged in PBS after isolation from whole blood and under standard cell culture conditions, the mass of CD3+ T cells remained unchanged for over four hours (FIGS. 3A and B). The collection of 20 phase and brightfield images each for all cells requires 90 minutes to complete.

### Data Analysis

Incubator conditions and hardware performance were monitored to ensure consistency during data collection; absence of fluctuations in temperature, carbon dioxide levels, and focus position were confirmed prior to image analysis. Phase images were processed using custom MATLAB programs designed to track individual cells between successive images and calculate their dry masses according to the equation m=k∫θλdA, where m=mass (picograms), k=5.56 pg/μm³, θλ=measured phase shift, and A=area (square microns), as demonstrated previously by Zangle, et al. The mass of each cell was calculated by taking the median of 20 individual mass measurements; cells whose measurements' coefficient of variations were greater than 15% were excluded from analysis. Cell masses for each patients' time points were also normalized to the median mass of their respective product sample by dividing each cell's mass by the median T cell mass of the infused apheresis product.

### Statistical Analysis

The relationship between normalized cell mass and acute GVHD was assessed over time using a generalized linear mixed model. The binary outcome acute GVHD (“Yes” or “No”) was modeled against a 4-level time categorical, time fixed effect (14 days, 30 days, 60 days, 100 days), a continuous fixed effect for normalized cell mass, and a fixed interaction effect. A subject-level random effect was included to account for within-subject dependence over time, which was modeled with an autoregressive correlation structure. The binary outcome was defined as a “Yes” for a time point if acute GVHD has occurred before that number of days (14, 30, 60 or 100, respectively). This model was then used to estimate the probability of developing acute GVHD by that time point. Additional analyses were conducted to assess any lagged relationship between normalized cell mass and acute GVHD occurrence. For these analyses, new outcomes were generated where the binary outcome was defined as a “Yes” if acute GVHD occurred within a certain amount of time after a given time point (30, 60 or 90 days). The same model as specified above was used to estimate acute GVHD probabilities at each time point. The GLIMMIX procedure in the SAS statistical software (version 9.4, Cary, N.C., USA) was used to fit the generalized linear mixed model, while the ggplot2 package in the R statistical software was used to generate line graphs of the estimated probabilities.

### Results

### Clinical Outcomes of Enrolled Patients

Patients enrolled in this pilot trial had a median follow up of 11 months (range: 0.27-15.7) following SCT. Of the patients that underwent allogeneic SCT, 9 (69%) developed either acute or chronic GVHD. Median onset of acute GVHD (n=7, 6 MUD recipients) and chronic GVHD (n=4, all MUD recipients) were at 58 and 126 days post-transplant, respectively. The incidence of grade 3-4 acute GVHD was 15% and the incidence of moderate to severe chronic GVHD was 23%. In two instances, chronic GVHD was preceded by acute GVHD. Relapse occurred in one MUD recipient and CMV/EBV reactivation developed in nine and five (6/3 MUD, 4/1 MRD) patients, respectively. Seven of eleven MUD recipients are surviving, as are all MRD and autologous SCT recipients. Post-transplant, two patients diagnosed with GVHD later died; after suffering multiple infections, one patient that remained GVHD-free died.

### T Cell Engraftment

T cell chimerisms were measured by PCR for short tandem repeat sequences in donor and recipient DNA isolated from T cells. Average T cell chimerism for allogeneic transplant recipients was 90% at 30 days post-transplant (T30), 99% at 60 days post-transplant (T60), and 99% at 90 days post-transplant (T90). All patients that remained GVHD-free achieved 100% chimerism by T30. Average T cell chimerism for patients that developed GVHD were 87% (T30), 99% (T60), and 99% (T90).

Donor-derived CD3+ T cell (ddCD3) counts were calculated as previously reported (Toor A A, et al. Biol Blood Marrow Transplant. 2012; 18(5):794-804). Average ddCD3 cell counts were 571 cells/μl, 1417 cells/μl, and 1019 cells/μl at 30, 60, and 90 days post-transplant, respectively. Median ddCD3 counts were generally slightly lower in patients who developed GVHD (T30=117 cells/μl, T60=1140 cells/μl, T90=1110 cells/μl) relative to patients that remained GVHD free (T30=3792 cells/pi, T60=590 cells/μl, T90=1350 cells/μl).

### T Cell Mass Measurements

Donor apheresis product study samples obtained for T cell mass measurements yielded an average of 1552 CD3+ T cells for imaging and analysis, while recipient whole blood samples each yielded an average of 590 cells (n_{T14}=400 cells, n_{T30}=657 cells, n_{T60}=605 cells, n_{T100}=709 cells). The average median mass of CD3+ T cells in donor apheresis products was 60 pg, while recipient whole blood samples yielded T cell populations with an average median mass of 69 pg (T14=73 pg, T30=71 pg, T60=67 pg, and T100=67 pg).

As shown in Table 2, the average median mass of T cells from infused stem cell products was higher (68 pg) for autologous SCT recipients than for allogeneic SCT recipients (58 pg). Similarly, a higher average CD3+ T cell median mass (64 pg) was observed in infused product samples for patients without GVHD compared to patients that developed GVHD (57 pg). Infused product samples for matched-related donors (median CD34 cell dose=4.49×10⁶ cells/kg) yielded an average T cell median mass (64 pg) larger than that of matched-unrelated donors (median CD34 cell dose of 4.59×10⁶ cells/kg) (57 pg).

TABLE 2
Average T Cell Masses by Time Point.
A Average CD3+ median mass in picograms by time point

| | Product | T14 | T30 | T60 | T100 |
|---|---|---|---|---|---|
| ALL (n = 17) | 60 | 73 | 71 | 67 | 67 |
| ALLO (n = 13) | 58 | 72 | 72 | 67 | 66 |
| AUTO (n = 4) | 68 | 73 | 70 | 69 | 71 |
| NO GVHD (n = 7) | 64 | 74 | 70 | 67 | 68 |
| GVHD (n = 10) | 57 | 71 | 72 | 67 | 66 |
| MRD (n = 2) | 64 | 71 | 77 | 67 | 66 |
| MUD (n = 11) | 57 | 73 | 71 | 67 | 66 |

B Average normalized CD3+ median mass by time point

| | Product | T14 | T30 | T60 | T100 |
|---|---|---|---|---|---|
| ALL (n = 17) | 1.00 | 1.17 | 1.20 | 1.15 | 1.14 |
| ALLO (n = 13) | 1.00 | 1.21 | 1.26 | 1.17 | 1.17 |
| AUTO (n = 4) | 1.00 | 1.08 | 1.03 | 1.07 | 1.01 |
| NO GVHD (n = 7) | 1.00 | 1.16 | 1.10 | 1.09 | 1.05 |
| GVHD (n = 10) | 1.00 | 1.18 | 1.29 | 1.19 | 1.18 |
| MRD (n = 2) | 1.00 | 1.12 | 1.21 | 1.05 | 1.04 |
| MUD (n = 11) | 1.00 | 1.24 | 1.27 | 1.19 | 1.19 |

A Average CD3+ median mass (picograms) by time point for patients grouped by transplant type, GVHD status, and donor type.
B Average normalized CD3+ median mass by time point for patients grouped by transplant type, GVHD status, and donor type

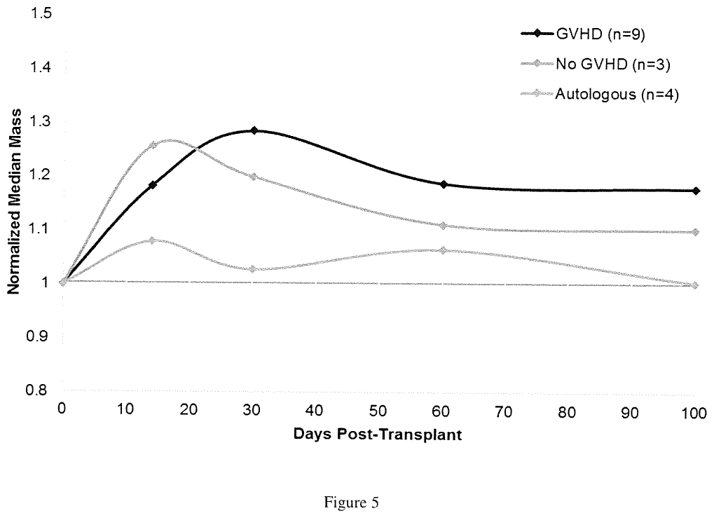

Post-transplant variability in T cell masses was observed in allogeneic SCT recipients (FIGS. 4A and B). Those who did not develop GVHD exhibited an initial (day 30) departure from, and later (day 60) restoration of T cell mass to levels similar to the infused stem cell product. Autologous SCT patients displayed similar kinetics (FIG. 5). Together, autologous and allogeneic SCT patients free of GVHD exhibited no significant difference between infused product and day 100 normalized median T cell masses (p=0.30, Student's t-Test).

Conversely, patients with GVHD exhibited a significant (p=0.006, Student's t-Test) difference between product and day 100 normalized median masses, showing a persistent elevation (˜18% higher median T cell masses than infused stem cell product) beyond day 30. Five patients exhibited a 20% or greater increase in median T cell mass at day 60 or 100, two of whom developed grade III-IV acute GVHD and one, severe chronic GVHD (FIG. 5). One of these patients, a 24-year-old male with Hodgkin's Lymphoma, suffered Grade III acute GVHD and exhibited normalized median T cell masses of 32% (day 60) and 25% (day 100). Another, a 65-year-old male with Myelodysplastic Syndrome, was diagnosed with severe chronic GVHD and exhibited median T cell masses 36% and 45% greater than infused stem cell product cells at days 60 and 100, respectively. Both patients later died.

### T Cell Mass-Time Interaction

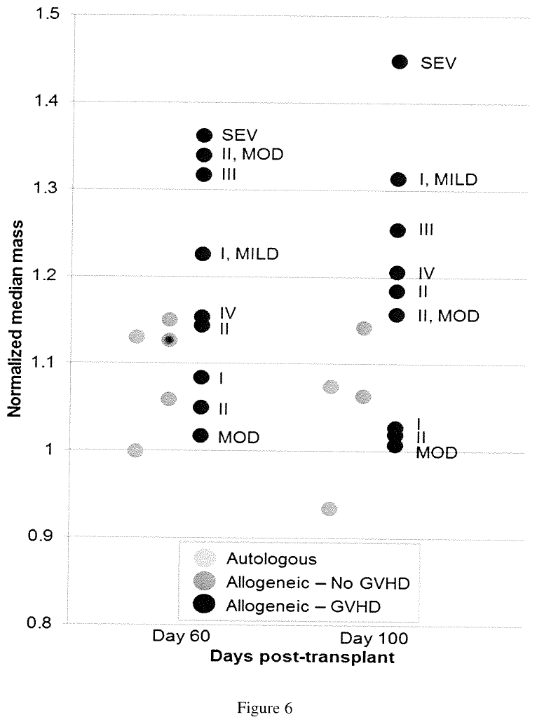

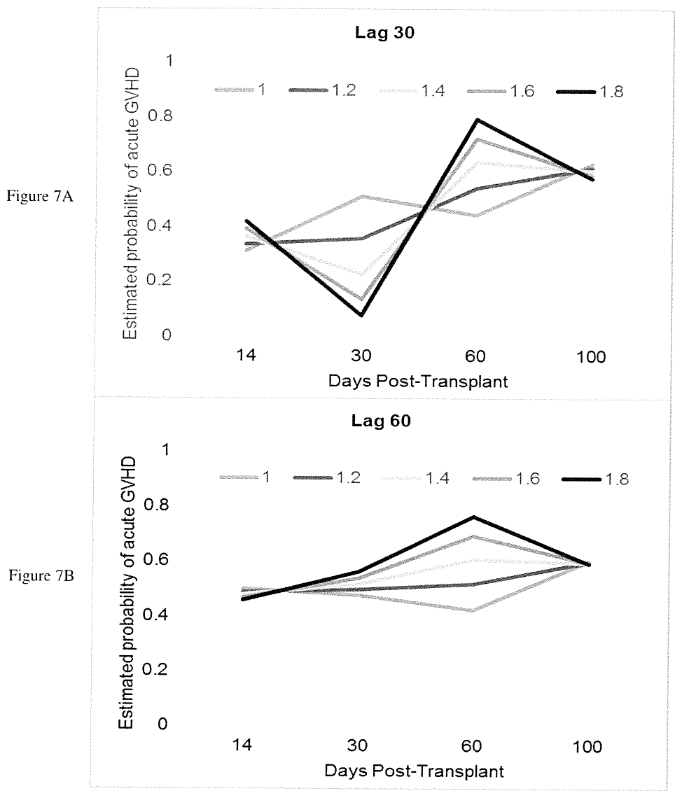

Studying the T cell mass and time interactions using generalized linear mixed modeling did not reveal a significant interaction effect between normalized cell mass and time (p-value=0.7791), nor significant main effects of time (p-value=0.7793) or normalized cell mass (p-value=0.7135) on acute GVHD occurrence. While not significant, FIG. 6A shows that there is a positive relationship between normalized cell mass and the probability of acute GVHD occurrence at 60 days (probability of acute GVHD occurring within the next 30 days increases as normalized cell mass increases). Given the small sample size, this series of analyses did not reveal any other T cell mass associations with estimated acute GVHD probability based on normalized cell mass at 14 or 100 days.

### Discussion

The normalized mass data obtained in this study showed that patients who developed GVHD had, on average, T cells with larger normalized masses at 60 and 100 days post-transplant relative to GVHD-free patients. Of patients with GVHD, individuals exhibiting median masses 20% or greater than infused stem cell product at days 60 or 100 tended to have more severe cases of both acute and chronic GVHD. Conversely, the kinetics of GVHD-free patients more closely resembled those of autologous transplant recipients, showing a return of normalized mass to levels similar to infused donor products by day 100. Interestingly, no relationship between absolute mass and GVHD status was observed. As no studies have characterized the variability of T cell population masses between individuals, it is possible that T cell heterogeneity contributes to this observed absence.

Relative to other potential biomarkers and detection techniques, quantifying T cell mass using LCI provides a rapid, inexpensive, and comprehensive measure of T cell activation. The assessment of T cell population mass may be used to predict alloreactivity and, subsequently, to guide the titration of immunosuppression in allogeneic SCT recipients.

### Example 2. Use of High-Speed LCI (HSLCI) to Characterize Biomass Kinetics of Melanoma Cell Lines

This example describes a multi-center study using high-speed LCI to characterize biomass kinetics for three isogenic sensitive/resistant pairs of patient-derived, V600E-positive, melanoma cell lines in response to the BRAF inhibitor Vemurafenib. We demonstrate that the LCI-determined biomass kinetic signatures taken 24 hours after dosing can be used to discriminate between drug-sensitive and drug-resistant populations. These LCI data are shown to be reproducible between study sites, and in-line with traditional multi-day cell counting growth inhibition assays. Our results highlight the use of high-speed LCI as a reliable tool for rapid measurement of drug resistance in melanoma.

### Materials and Methods

Cell counting. Cells in multiple six-well tissue culture plates were incubated overnight at 37° C., 5% CO₂). Each six-well plate contained one well with a media control, one well with 0.1% DMSO vehicle control, and four wells of Vemurafenib at 1 uM, 3 uM, 5 uM, or 10 uM concentrations. Cells were counted each day for five days following seeding.

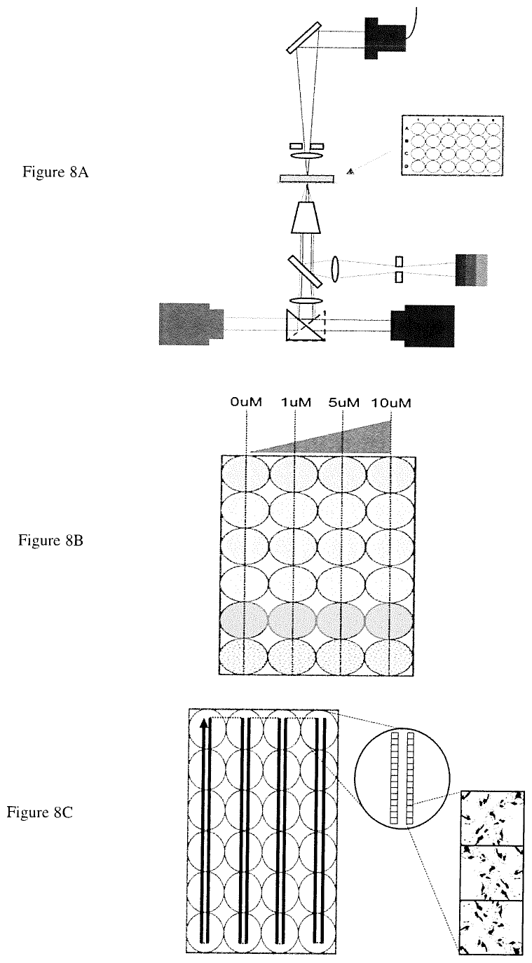

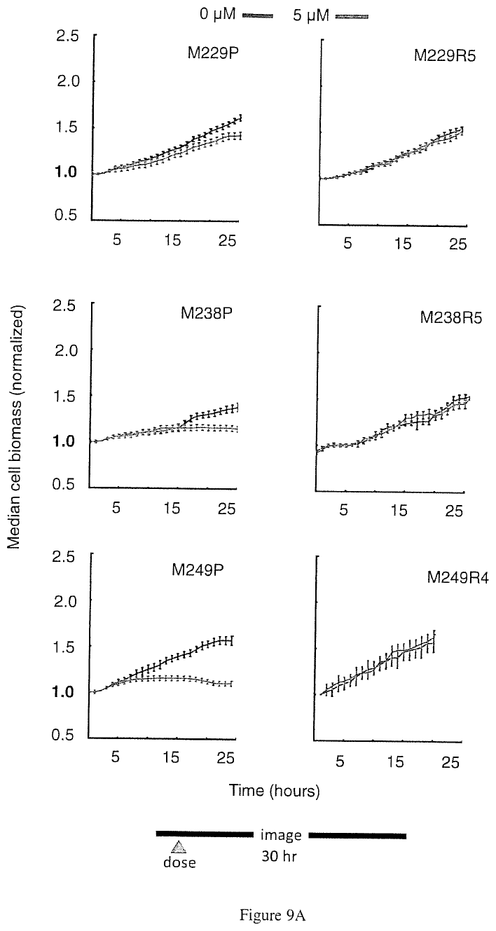

Live Cell Interferometry. The standard and high speed LCI platforms were based on a custom built inverted microscope, coupled to a quantitative phase detecting camera (SID4BIO, Phasics) which consists of a modified Shack-Hartman mask, fitted to a 1600×1200 pixel CCD camera [Imprex B1621]). The phase calculation down samples the raw image to 400×300 pixels, resulting in effective pixel sizes between 1.3 μm and 2.5 μm, depending on the objective. We utilized frame rates between 4 fps and 10 fps; maximum imaging throughout is limited in practice by the time required to calculate the phase image (typically 500 ms on an Intel Core i7 processor) and post processing (flattening, cell segmentation, etc., described below; typically 500-1000 ms on a single core). The growth kinetics and population mixing studies utilized a 20× objective (Nikon Neoflaur, NA 0.5 or Nikon Plan Fluorite, NA 0.3), while the high speed LCI studies utilized a 10× objective (Nikon Plan Fluorite, NA 0.3) to obtain larger fields of view. FIG. 8A shows a schematic of the high speed LCI system. In both the standard and high speed systems, position was determined by maximizing contrast during a vertical scan though focus. The high speed LCI was equipped with a custom coaxial optical beam deflection position sensor coupled with a feedback loop to automatically hold the objective in position of best focus during lateral scanning. During lateral scanning of the multi-well plate, the illumination was strobed to coincide with the camera exposure, using a trigger generated by the B1621 at a rate of 4 fps. Typical time to scan one column of six wells was 60 seconds. During this time, 30 images are taken in each well. The two test sites differed in the use of either stage-top incubation (UCLA site; Ibidi) or placing the entire system in a standard cell culture incubator (VCU site) to maintain cell viability. Both schemes proved equivalent in our tests.

Image analysis. Raw interferograms were converted to phase images using the manufacturer's software and analyzed with our custom MATLAB script (The MathWorks). The image processing pipeline has been described in detail previously. [Reed, J., et al., Rapid, massively parallel single-cell drug response measurements via live cell interferometry. Biophys J, 2011. 101 (5): p. 1025-31.] Briefly, the computed phase images were first corrected for low frequency background noise inherent to the shearing interferometry method. Using the resulting flattened phase images, single cells segmented from the background using Canny spatial-derivative edge detection algorithms, their locations, and optical volumes were recorded. Frame-to-frame cell tracking was accomplished with a particle tracking algorithm IDL particle tracking. [see the website located at www.physics.emory.edu/faculty/weeks//idl/. Single cell growth tracks were quality filtered using an upper cutoff of +1-15% uncertainty (s.d. of residuals) in the calculated growth rate, as determined by linear fitting the biomass versus time data. Compared to our prior implementation, code was optimized for fast execution speed, and near real-time processing was achieved by adding additional processor cores.

Population kinetic response experiments. Cells were synchronized by growing to confluence in 75 mL tissue culture flasks and collected using a “shake-off” technique. Cells were plated at 1×10⁵ cells/ml in 25 mm dishes and incubated overnight. Prior to imaging, samples were equilibrated thermally for one hour on the HSLCI and LCI stages, then imaged for three hours, after which either 0.1% DMSO vehicle control or 5 uM Vemurafenib was administered and dishes imaged for another 25-30 hours.

Fluorescence mixing experiments. 1.25×10⁴ M249P and GFP-M249R4 cells were added together in a total volume of 1 mL tissue culture media. 0.7 mL of the mixture was dispensed into each well of an Ibidi 4 well Ph+ μ-slide. Cells settled over 6 hours after which 5 μM Vemurafenib was added to the wells. Ibidi oil sealed the liquid opening of each well before the plate was put onto the LCI stage. All 4 wells were imaged continuously for 48 hours.

Vemurafenib dose response experiments. Cells were synchronized by shake-off, seeded into four wells each of a 24-well glass bottom plate at 1×10⁵ cells/ml, and incubated overnight. Cells were dosed with 0.1% DMSO carrier control, or 1 uM, 5 uM, or 10 uM Vemurafenib. Cells were incubated for 24 hours, then imaged on the HSLCI for 10 hours.

Kinase inhibitor screen. 1× Dose Determination: For each inhibitor, a 1× dose was calculated. Briefly, recommended clinical doses were obtained from published package inserts. Doses were matched to peak serum concentrations (Cmax (ng/mL)) for each inhibitor as measured in clinical trials in which the recommended doses were utilized. Using each drugs' molecular weight, peak serum concentration values were converted to uM units and designated as 1×. Drug Sourcing and Preparation: Imatinib (Cat. #S1026), Vemurafenib (Cat. #S1267), Dabrafenib (Cat. #S2807), Selumetinib (Cat. #S1008), Trametinib (Cat. #S2673), Cobimetinib (5 mg, Cat. #58041) and Binimetinib (10 mg, Cat. #57007) were purchased from Selleck Chemicals. Imatinib, Vemurafenib, Dabrafenib, Selumetinib, and Trametinib were supplied at 10 mM in DMSO. Cobimetinib and Binimetinib were supplied as dry powders and suspended in DMSO to a final stock concentration of 10 mM upon receipt. Cell Preparation and Dosing: M249R4 cells were plated in a 24-well optical glass-bottomed plate (Cat. #P24-0-N, Cellvis) at 1×10⁴ cells/ml (total of 1 mL in each of 24 wells) in media (DMEM, 10% Fetal Bovine Serum, 2 mM L-Glutamine) containing 1 uM Vemurafenib. Plated cells were allowed to adhere overnight at 37° C., 5% CO₂. All cells were washed with 1× phosphate-buffered saline, pH 7.4, and provided with fresh media. Immediately following washing and feeding, cells were dosed with inhibitors at dose-escalating concentrations and incubated under standard cell culture conditions for 24 hours. After incubation, cells were imaged for 10 hours using the HSLCI system.

### Results

### BRAF-Inhibitor Sensitive and Resistant, Paired Melanoma Cell Lines

Up to 50% of melanomas contain activating mutations in the MAPK signaling pathway BRAF serine/threonine protein kinase. This study evaluated three BRAF V600E mutant lines (M229, M238, and M249), sensitive to BRAFi Vemurafenib (IC50<1 uM), and their isogenic, resistant companion lines, created by co-incubation over time with Vemurfenib. (Table 3). These three BRAFi sensitive and resistant paired melanoma lines, with characterized molecular lesions that enable drug-targetable mechanisms of resistance, provide a unique resource for evaluating LCI platform quantification of biomass growth responses to targeted therapies and combinations. Molecular studies show that Vemurafenib-resistant M229 (‘M229 R’) and M238 (‘M238 R’) cells upregulated the surface PDGFRβ to defeat frontline therapy. M229 R and M238 R lines resist death by increased p-ERK, p-AKT, and p-p70S6K levels, providing a MAPK-redundant, hyperactive-AKT survival pathway that was sensitive to combinatorial drugs targeting the PI3K-AKT-mTORC signaling axis in standard bulk growth assays. In contrast, M249 R cells acquired a N-RAS (Q61K) mutation to bypass BRAF inhibition, but still remain sensitive to downstream MAPK signaling pathway inhibitors that target MEK1/2.

TABLE 3
Patient-derived melanoma lines.

| Cell Line Designation | Molecular Lesion(s) | Efficacious Agents |
|---|---|---|
| M229 | BRAF V600E | BRAF inhibitors |
| M229 R | BRAF V600E, PDGFRb upregulation | BRAF + PI3K/AKT/mTORC inhibitors |
| M238 | BRAF V600E | BRAF inhibitors |
| M238 R | BRAF V600E, PDGFRb upregulation | BRAF + PI3K/AKT/mTORC inhibitors |
| M249 | BRAF V600E | BRAF inhibitors |
| M249 R | BRAF V600E, N-RAS Q(61)K | BRAF + MEK1/2 inhibitors |

### Live Cell Interferometry

Previous LCI studies provided proof-of-principle validation for targeted, single agent small-scale work, such as Herceptin sensitivity in breast cancer and tunicamycin activity in multiple myeloma. However, multi-agent, multi-concentration screening for rapid interrogation and therapy selection using patient samples will require a substantial increase in the number of tumor cells analyzed per hour. To achieve this forward-looking goal, we constructed a high throughput screening LCI (HSLCI), utilizing industrial-grade imaging hardware and accelerated automated image analysis and data processing pipelines using low-cost, multi-core PC processors and additional software improvements. We built a HSLCI using a custom inverted microscope system equipped with a modified Shack-Hartman wave front sensing camera for phase measurements and standard wide-field CCD camera for bright field and fluorescent imaging (FIG. 8A). Stage-top or whole microscope enclosures provide long-term environment stability for imaging under physiology-approximating conditions (37° C., 5% CO₂). The HSLCI images up to 24 individual wells simultaneously and each well can contain a different cell type or line exposed to a unique drug dose or combination (FIGS. 8B and C). Details of LCI and HSLCI platforms and implementation differed slightly between the two test sites (See Methods).

Biomass Kinetic Responses with Vemurafenib Exposure

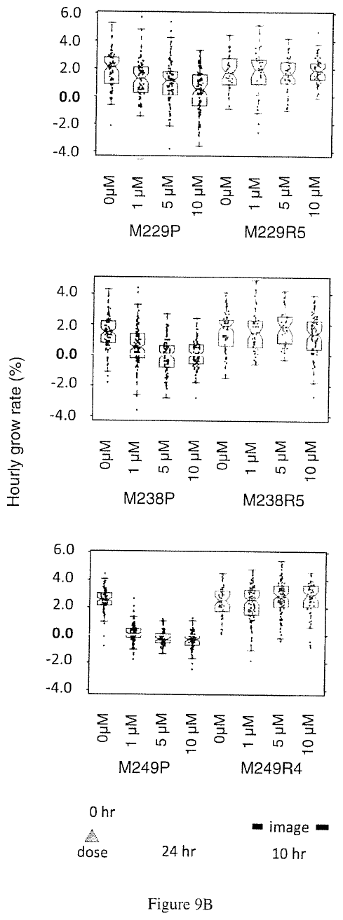

There is no existing data for the rate of biomass change of BRAFi sensitive or resistant melanoma cells that grow as adherent single-cells or clumps. Therefore, we measured the kinetics of Vemurafenib response in the three paired, molecularly characterized melanoma lines using standard and high speed LCI, to establish rates and distributions of biomass change with or without drug exposure. First, we performed a standard multi-day dose-escalation cell-counting assay to confirm sensitivity for the three parent and matched resistant lines at 1.0 μM to 10.0 μM Vemurafenib exposure (not shown). As anticipated, the parent lines slowed and the matched resistant lines continued replicating with drug exposure. We next used 5 μM Vemurafenib as the mid-point drug dose to measure the median population growth rate by HSLCI for the six cell lines in the first 25-30 hours of drug exposure, in order to quantify the average population kinetic response. (FIG. 9A) Drug sensitivity of M249P cells was detectable as early as six hours, and significant growth rate reduction occurred in all three parental lines by 20 hours using HSLCI. Plotting the growth rate distribution obtained by LCI for each hour of 504 Vemurafenib exposure showed the extent of population growth rate heterogeneity in each sample and a decline below zero growth for most M249P cells, in contrast with M249R4 cells that almost all retained a net-positive growth rate (not shown). These results reproduced independently at both experiment sites with standard and high speed LCI platforms.

The mid-point drug data suggested that Vemurafenib sensitivity, or lack thereof, would be distinguishable in a drug-escalation assay as for cell counting by measuring changes in sample growth rates after 24 hours of drug exposure. To test this hypothesis, and to establish the HSLCI methodology for multi-dose/multi-agent screening, we collected short-term, 10-hour growth rate measurements of all three cell line pairs in parallel, at escalating Vemurafenib doses, using a 24-well format. All six melanoma cell lines were placed in a 24-well plate and dosed with 0.1% DMSO, or 1 uM, 5 uM, or 10 uM Vemurafenib. The parental lines (M229P, M238P, and M249P) showed a clear pattern of increasing growth inhibition at escalating drug concentrations, whereas the resistant lines (M229R5, M238R1, and M249R4) showed no growth inhibition over the drug dosing range compared to vehicle DMSO controls, consistent with cell counting assays (FIG. 9B). We used Receiver Operator Characteristic (ROC) analysis to determine the ability to distinguish individual resistant cells from sensitive cells from changes in their individual growth rates during exposure to Vemurafenib. Both M229P and M238P lines were distinguishable from their resistant derivative lines at Vemurafenib doses of 5 uM (area under the curve (AUC), 0.60 and 0.85, respectively) and 10 uM (AUC, 0.78 and 0.75, respectively). The M249P line was most sensitive to drug and easily distinguishable based on changes in growth rate, with AUC greater than 0.90 at Vemurafenib doses of 1 uM and above.

### Kinase Inhibitor Screen in Vemurafenib-Resistant Melanoma

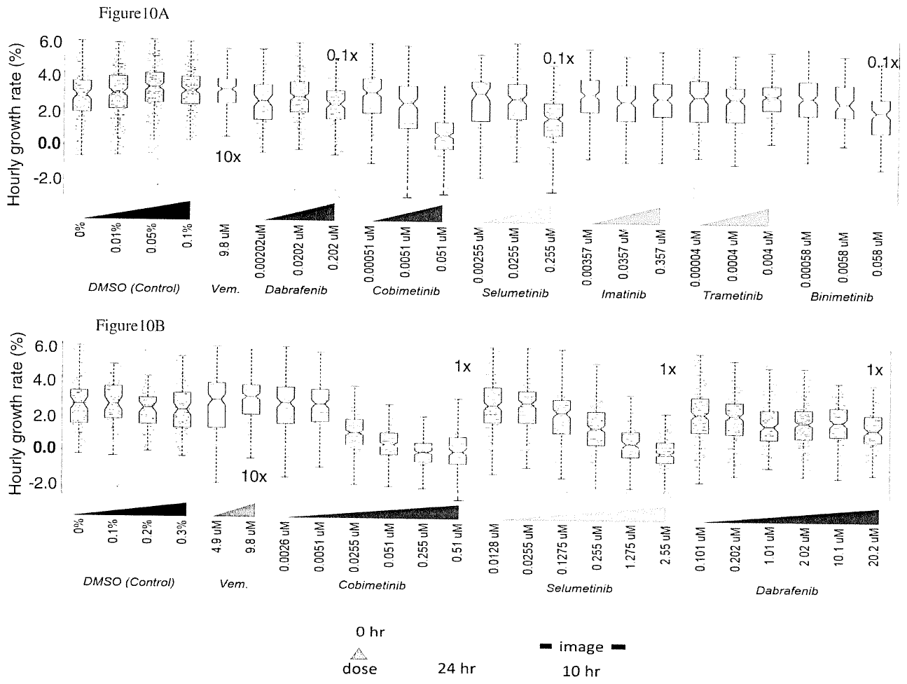

We performed high speed LCI dose response assays in triplicate using a panel of seven FDA-approved kinase inhibitors tested in clinical trials for treating metastatic melanoma to simulate selection of salvage therapy for a patient who had developed resistance to front line Vemurafenib (FIGS. 10A and B). These inhibitors target a spectrum of kinases (Table 2). We selected M249R4 cells for this study because of its robust growth profile and its strong resistance to Vemurafenib. For each targeted kinase inhibitor, a 1× dose was calculated to match the peak tolerated serum concentration (Cmax (ng/mL)), as measured in clinical trials. Our first assay screened all seven inhibitors at several concentrations to identify those that elicited the most apparent reduction in cell growth rates. The second assay used three of the inhibitors and a finer dosing gradient to demonstrate the sensitivity of the HSLCI approach for identifying a desirable dose response.

TABLE 2
Kinase inhibitor 1× dosing chart

| | | | | | | Serum Conc. | uM |
|---|---|---|---|---|---|---|---|
| Trade | Drug | Chemical | Clinical Dose | | Target | (ng/mL) | (1×) |
| Gleevec | Imatinib | STI571 | 400 | mg once daily | BCR-ABL, C-KIT | 2097 | 3.56 |
| Zelboraf | Vemurafenib | PLX4032 / RG7204 | 960 | mg twice daily | BRAF | 4800 | 9.80 |
| Tafinlar | Dabrafenib | GSK2118436 | 150 | mg twice daily | BRAF | 1050 | 2.02 |
| Cotellic | Cobimetinib | GDC-0973 / RG7420 | 60 | mg once daily | MEK1 | 273 | 0.51 |
| N/A | Selumetinib | AZD6244 / ARRY-142886 | 75 | mg twice daily | MEK1 | 1165 | 2.55 |
| Mekinist | Trametinib | GSK1120212 | 2 | mg once daily | MEK1/2 | 22.2 | 0.04 |
| N/A | Binimetinib | MEK162 / ARRY-162 | 45 | mg twice daily | MEK1/2 | 257.0 | 0.58 |

In a first assay, untreated control cells showed a median growth rate of 2.5% per hour. Cells treated with Vemurafenib grew slightly faster than the untreated control, consistent with prior studies that revealed the emergence of drug addiction. The median growth rate for all cells dosed with 0.001× and 0.01× concentrations of all seven inhibitors remained above 2% per hour. Importantly, at 0.1×, Cobimetinib and Selumetinib, each targeting MEK1, reduced M249R4 growth rates well below 2%. Binimetinib-exposed cells, targeting MEK1/2, also achieved a slight dose response. Dabrafenib, a BRAFi, caused a slight reduction in growth rate at 0.1×, whereas Imatinib and Trametinib, targeting BCR-ABL/C-KIT and BRAF, respectively, did not elicit responses when dosed at the same concentration (FIG. 10A). In a second assay, four inhibitors with activity at 1× or less were tested using a finer range of drug concentrations (FIG. 10B). Untreated control cells exhibited a median growth rate of 2.5% per hour and cells treated with Vemurafenib grew slightly faster than the untreated control, as anticipated. DMSO concentrations above 0.3% by volume appear to reduce growth. Both Cobimetinib and Selumetinib show robust dose responses over a dosing range of 0.05×-1×. Interestingly, Dabrafenib reduced growth rates at doses greater than 0.1×, but does not elicit a dose response nor does it kill cells up to a 10× concentration.

This Example demonstrates the ability of standard and high speed LCI platforms to rapidly quantify single cell drug sensitivity in tumor cell populations. This quantification may provide critical data for treatment selections on a whole-tumor population level and can identify specific subpopulation drug sensitivities to predict drug resistance at a single cell level. Our results also show the reproducibility of two similar but distinct LCI platform techniques by obtaining concordant data from two institutions. This provides confidence that the newly configured HSLCI has the required consistency for further development towards a clinically useful approach. This is important because the HSLCI is a more efficient approach compared to standard LCI and is capable of higher throughput and greater than 10-fold faster imaging speed, making it possible to examine many tumor samples in response to combinations of drug treatments at the same time.

Compared to conventional methods of tumor profiling that rely on circulating tumor DNA, circulating tumor cells, proteomic profiling using mass spectrometry, and chemosensitivity assays, the HSLCI has advantages of reduced cost, no labels, short turnaround time, and multi-parameterized outcomes. Furthermore, drug resistance assessments take about 24 hours, compared to 3-7 day turnaround times for equivalent chemosensitivity assays, which is ideal for primary patient samples. The acquisition of single cell drug responses in a heterogeneous population is a clinically relevant indicator for whole tumor response and drug resistance predictions. This ability improves therapeutic selection and helps improve patient outcomes.

HSLCI applications are not limited to melanoma and may find utility for almost any cancer including liquid discohesive tumors such as lymphoma and leukemia. Overall, our study highlights the use of HSLCI as therapy selection tool and for dissecting overall tumor heterogeneity, predicting single cell drug resistance and predicting treatment outcomes.

### Example 3. Quantitative Analysis and Process of High Speed Live Cell Interferometry Measurements

Live cell interferometry techniques allow the user to measure the phase shift of incident light through cells, which directly correlates to the cells dry mass. The prior art describes the measurement of dry mass of two cell samples with a 4×4 imaging grid of each sample using a quadri-wave lateral shearing interferometer (QWLSI) with z-ramp based focus. When a laser autofocus component is incorporated, it becomes possible to examine three times the sampling grid per sample in a fraction of the time. This new technique allows 24-48 samples to be measured continuously in parallel for 1-72 hours.

A problem with the previous LCI setup was the time it took to insure that the camera was in focus. Ideally, if minimal time lapse between frames is desired, the stage needs to run continuously. This would then require that the objective stay the desired distance away from the sample, even if the well shape/thickness/height changed. This requires a feedback loop that monitors the distance between the objective and sample and the ability to make any necessary changes to ensure the distance remains constant.

An optical beam deflection provides necessary sensitivity (˜100 nm) and speed (<100 Hz). Using a series of beam splitters, a laser beam can be reflected off of the glass to water interface where the cells are in suspension. A change in distance between the objective and surface gives a change in distance between the beam going out of the objective and the beam re-entering the objective. A signal based on this difference is transmitted to a sensor such as a quadrant photodiode (QPD) where the intensity/position of the laser is mapped to a dynamic analog voltage. Once this signal is conditioned, it serves as the input to a feedback loop. Since the distance between sample and objective is the key factor, the output of the feedback loop is accomplished by attaching the objective to a one-dimensional piezo stack with a range of 100 microns. The control of this feedback loop was accomplished using a stand-alone, low-cost microprocessor, Arduino Nano.

The control loop feedback used is a form of a proportional-integral-derivative (PID) controller where an error value is continuously calculated as the difference between a set-point and the measured process variable. Only the first two terms are currently being used, therefore the sum of the P and I terms make up the given output. The P term is the proportional gain times the error of that particular loop and the I term is the integral gain times the sum of error since the PID controller was started. Hence, the mathematical form is

output=K_{P}⊕error(t)+K_{I}≡error(|)d|.

In the application of scanning a plate, the I term handles small error as a function of time and the P term handles large errors.

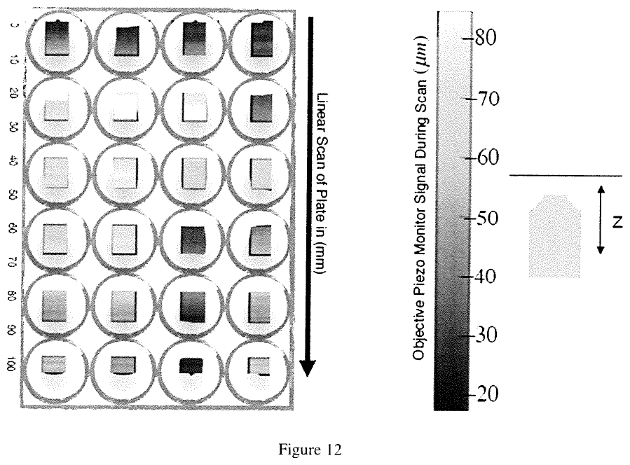

In its current high speed set-up, at any magnification, the stage scans at a maximum velocity of 2 mm per second while acquiring 4 frames, giving a spacing of one frame per 0.5 mm. The approximate time it takes the stage to make one pass across six samples covering a distance of 100 mm is 52 seconds. Once this pass is done, the data is saved to disk and the stage scans back through each well after a 1 mm step in the perpendicular direction. FIG. 12 shows the relative objective position for a typical scan. A video file with 210 frames is collected and parsed to the frames in the middle of each well. In this two minute span a 2×25 grid, in each well, is captured. The overall measurement and space between frames is dependent on the magnification. The normalized average pixel intensity of the interferogram per frame is determined and the peak intensity at the center of each well is calculated. The frames used are chosen as those with an intensity at least 60% of the peak frames. This faster imaging rate means the data will need to be processed faster. The capturing and logging of these videos are done with a capture computer (CC) and processing is done on a separate computer, a processing computer (PC). The PC is linked to the CC e.g. via a standard cat5 ethernet cord on a local network. This can be limiting depending on the desired turnaround time from capture to usable data considering the video file, and for this experiment, is approximately 0.5 GB of data. If each video file takes 52 seconds to capture and write to disk, a file can be pulled and processing begins immediately. The parsing of the frames is done in one step where the video file is read from a disk on the CC and then written as a data file that contains an individual frame via the PC. Because this is done in real time, a custom Matlab code is needed to constantly compare the processed video files with the unprocessed video files. To ensure that the data files constructed from each video files are labeled correctly, the necessary location information is pulled from the video file first.

As each interferogram is pulled from the video file, it is converted into the flattened phase image before moving to the next. At the moment, this takes the most time. For this example, approximately two second per frame were needed to develop the phase image. Given that this is most time consuming process, this is the only procedure done during the experiment. The remaining process of tracking the cells is done after the experiment. The custom Matlab code first defines what is and what isn't a cell by a predefined range in both optical thickness and area. It tracks the x and y position, mass and area of the cell, and compares the same metrics of the remaining frames from the same location. If these values do not vary more than a given threshold between frames, it is tagged and tracked as a cell.

The camera/interferometer used in the present example was a quadriwave lateral shearing interferometer (QLSI). Use of this camera does not require labeling of the cells (is label-free) and provides additional phase information about the refractive index of each specimen. The refractive index distribution across the imaging plane creates contrast in the interferogram.

The advantage of a shearing interferometer is its self-reference capability. This allows for a simple and compact system that is not as sensitive to vibrations or other external noise. For the present example, the interferogram was created by a diffractive grating mounted to the front of a CCD camera, where an incident beam is diffracted into four replicates. The replicas then interfere on the surface of the camera where the interferogram is recorded.

To obtain the unwrapped phase image, the intensity signal across the image matrix is de-convolved in the Fourier domain around the spatial period of the grating. This produces a phase gradient or map that is then numerically integrated to get the optical path difference.

The useful measurement made by the interferometer is the optical path difference and is to defined as a function of the spatial position in the wavefront. Thus

$$OPD ( x , y ) == {{\int }_{o}}^{h} [ n ( x , y ) - {n}_{n , Δ um} ] dz$$

Here n is the refractive index of the specimen and [symbol] is the refractive index of the medium around it. The difference is integrated over the total thickness h in the direction of propagation. This value is a combination of OPD from the specimen and the OPD from the imaging system, however we eliminate any contribution from the imaging system by subtracting the reference image captured before any measurements are made.

The OPD is then used to find the optical volume difference OVD, which is directly proportionate to the dry mass of the cell by a constant known as the specific refractive increment a. The specific refractive increment is the rate of change in the refractive index n of a specific specimen. The mass is:

$$m = \frac{1}{α} S \cdot OPD$$

where S is the surface area of the specimen in microns. For the mass measurements made during the current experiments 1/α was defined as 5.56 pg/μm³.

FIG. 13 depicts the general beam path used in the present Example. The laser used was a CPS980, by Thorlabs. This laser has a wavelength of 980 nm and produces an elliptical beam shape roughly 3.8 mm×1.8 mm, with a power of 4.5 mW. It is particularly useful because it is compact at 11 mm in diameter and 40 mm in length and is engineered to withstand large temperature variations. The laser is mounted into an adjustable mount so that the position of the beam incident on the rear aperture of the objective can be controlled. This can be adjusted to insure there is an offset of the laser beam from the optical axis.

The first lens the laser is passed through is a bi-convex lens with a focal length of 500 mm. It is placed roughly 100 mm in front of the laser and 305 mm from the sample. This helps to reduce the elliptical shape of the laser beam at the sample. Once the beam passes through the first focusing lens, it passes through a polarizing beam splitter where s-polarized light is reflected and p-polarized light is transmitted. In combination with the proceeding half wave plate, this achieves two things. Since the laser diode has a polarization extinction ratio of 15 dB, nearly all of the laser is initially transmitted. The half wave plate is then used to rotate the polarization plane of the linearly polarized light. This is not as important for the beam from the laser as it is for the reflected beam.

The wave plate can be adjusted so that the returning beam can be reflected instead of transmitted through the beam splitter. This reduces any interference that the returning beam may cause to the laser diode itself, as well as reflects it into the photo diode. Once the beam is reflected off the polarizing beam splitter, it is focused again with another bi-convex lens that has a focal length of 50 mm. This lens serves to control the spatial resolution of the distribution in the intensity of the laser beam. By adjusting the distance between the lens and the photo diode, any diffraction effects can be masked from the different optical components.

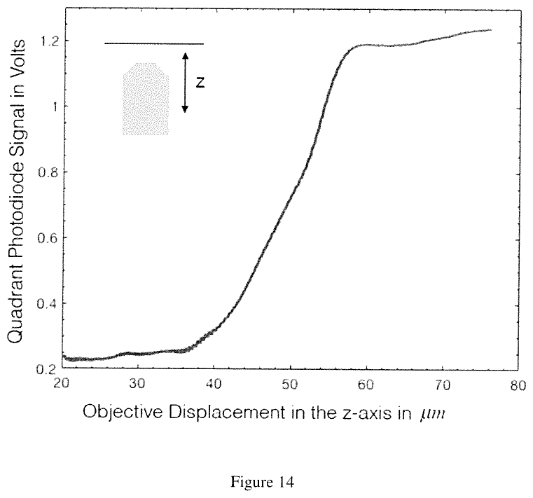

It is imperative to make any adjustments to the laser beam while the sample or water to glass interface is at the focal length of the objective and the piezo stack is in the middle of its range. There is a relative window where the displacement on the photo diode is linearly dependent on the distance between objective and sample. This window is much greater than the focal range of the objective but fine adjustments can be made to ensure the reduction of noise over this range. When configuring, or taking a pre-run quality check, the objective is made to ramp ±5 μm to both insure quality and to find the ratio between photo diode signal and the displacement in micron of the objective. For example, FIG. 14 shows a 40× objective that is ramped ±30 μm and the conditioned QPD signal in volts is plotted versus the displacement of the objective.

The key component in bringing together the laser optical path and the imaging path is a short-pass dichroic mirror with a cutoff of 805 nm. It spectrally separates any incident light by transmitting and reflecting it according to wavelength. Since the laser beam is in the near infrared range, 95% will be reflected. With the opposite effect, the imaging plane is transmitted allowing it to continue to the camera. The sample is illuminated with a 660 nm, 13 mW LED diode. The LED provides a spatially coherent light source that is optimal for the phase imaging process of the interferometer. When using higher magnification, a focusing lens is required to direct as much light into the objective as possible. Once the plane wave of the image passes through the dichroic mirror, they are focused with an imaging lens into the camera.

When designing a commercial or scientific device, it is advantages to find a balance between cost and efficiency of the electronic circuitry or hardware. Today's “open source” market offers a number of different options. For the present example, the user interface was done in the numerical computing environment MATLAB.

The interferometric camera employed in the present example uses a GigE Vision interface that is based on the Ethernet standard. With this connection, images/videos are transferred from the camera to the CC at a rate up to 10 Gbits/s; for this example, a 1GigE version was used. An advantage of the GigE Vision compared to other internet protocols is that a point-to-point connection is created to avoid interaction from external devices. This means that the user must configure the device, instead of automatic configuration found in other protocols, allowing a faster transfer rate and higher bandwidth. The camera is configured to receive a trigger signal that controls the fps. This was done with an Arduino nano, where the stage move trigger is configured to an interrupt pin on the nano to insure that the stage and camera are always in sync. At the end of each pass the video file is saved to disk and the camera is set-up for the next acquisition.

The motors that control the x, y, and z motions of the stage are linked to MATLAB via a USB COM port to a Thorlabs APT stepper motor controller. This controller uses a digital signal processor (DSP) and the ActiveX software framework to give high resolution micro-stepping. Like other in-process COM servers the Thorlabs APT ActiveX control is used as the server and

MATLAB is a control container or client. Therefore both a GUI and MATLAB scripts can manipulate the ActiveX controls' properties, methods and events. The motors are linked to the controller through 15-pin D-sub connectors that allow for encoding and 48V 2-phase bipolar motor outputs. The controller is also connected to the trigger generator with a 5V logic level output that generates a signal when the stage begins to move.

For the purpose of high bandwidth data signal acquisition and error analyses, a NI USB-6002 DAQ is connected to MATLAB using the MATLAB data acquisition toolbox. Using a session-based interface, a binary file including the camera trigger, amplified photo diode signal and position monitor of the piezo stack are saved to disk to accompany each video file.

The last component that is directly linked to the MATLAB interface is the Arduino DAQ. This setup uses a MATLAB support package that uses a server program running on the board to execute and receive commands via a serial port. This is a necessary connection between the PID controller and MATLAB for a number of reasons. The PID controller could not be directly connected because the support package only allows for control loops to run at up to 25 Hz. This is much slower that the capabilities of the Arduino and thus dramatically slowing down the refresh rate of the feedback loop. As the PID controller is the only thing controlling the position of the piezo, handshaking is done to go between ramp and PID functions. In addition to function switching the PID controller sends a signal to MATLAB via the Arduino DAQ that the piezo stack is at the top or bottom of its range. This then causes the stage to step in the z-direction to bring the piezo back into its operating range. The “handshaking” that is done between the Arduino DAQ and PID controller is achieved with multiple digital pins using TTL type logic. The PID controller is solely designed to process the input signal and define an output. Because the Arduino Nano has limitations in voltage range and resolution, the I2C bus line built into the Arduino is used to communicate with additional breakouts. The input signal from the photodiode sensor has a range of ±15 V so a signal conditioning circuit is needed to convert the signal into the desired range of [0-5V]. The analog input resolution for the Arduino nano is 8-bits where a higher resolution of 16-bits was accomplished by using the ADS1115 analog to digital converter (ADC) breakout made by Adafruit industries. This breakout is capable of 860 samples per second over the I2C bus line and only consumes 150 micro-amps of energy. On the same bus line, the MCP4725, a 12-Bit digital to analog convertor (DAC) also made by Adafruit is used to give a output voltage range from [0-5V]. The Arduino has built in ADCs on the analog pins, but the nano does not contain a DAC. Therefore the ADC was a bonus addition to the set-up while the DAC is more of a necessity. Downstream from the DAC is a 2× amplifier comprised of an op amp and two resistors. This brings the output voltage of the feedback loop into the [0-10V] range that the piezo controller is expecting.

The piezo controller that was used was a Nano Drive® and the piezo stack was a Nano-F100S, both by Mad City Lab. The controller runs in a closed feedback loop with the stack to ensure that the relationship between the input voltage and displacement of the piezo remains linear. This eliminates creep and hysteresis found in piezo actuators. The piezo stack uses internal position sensors to keep the error in linearity, over the full range, to <0.01%. The [0-10V] input is mapped directly to a [0-100 μm] range at the objective.

The optical beam detector that was used was a Thorlabs PDQ80A quadrant position detector. This sensors peak responsivity is at 900 nm which is in the IR range of the laser. The recommended laser spot size for this sensor is 1-3.9 mm to ensure that a significant amount of signal strength is not lost when the spot crosses the ˜0.1″ gap between any quadrant. This sensor is very sensitive to the shape and density distribution of the incident beam.

As seen in the ray diagram, a series of lenses were needed to minimize diffraction patterns (from the glass thickness of beam splitters and the dish itself) in the beam. If the beam is not focused to a small spot, the diffraction patterns will dominate during a ramp of the piezo stack. Essentially the beam needed to be focused down to the point where the distance between each peak was smaller than the resolution of the diode. This gives a beam, whose distribution within the beam as well as the beam, moved side to side. The final desired beam size was measured at ˜0.75 mm.

The change in voltage coming directly out of the QPD during a ±5 μm ramp is ±25 mV. Therefore, to condition and amplify the signal an instrumentation amplifier was used. The amplifier comprises multiple components, e.g. the gain/buffer is accomplished with a standard op-amp circuit; a low-pass filter (e.g. with an attenuation of 6 dB at 30 Hz); and a biasing circuit. These elements can be adjusted to accommodate any noise coming from the electronics or environment.

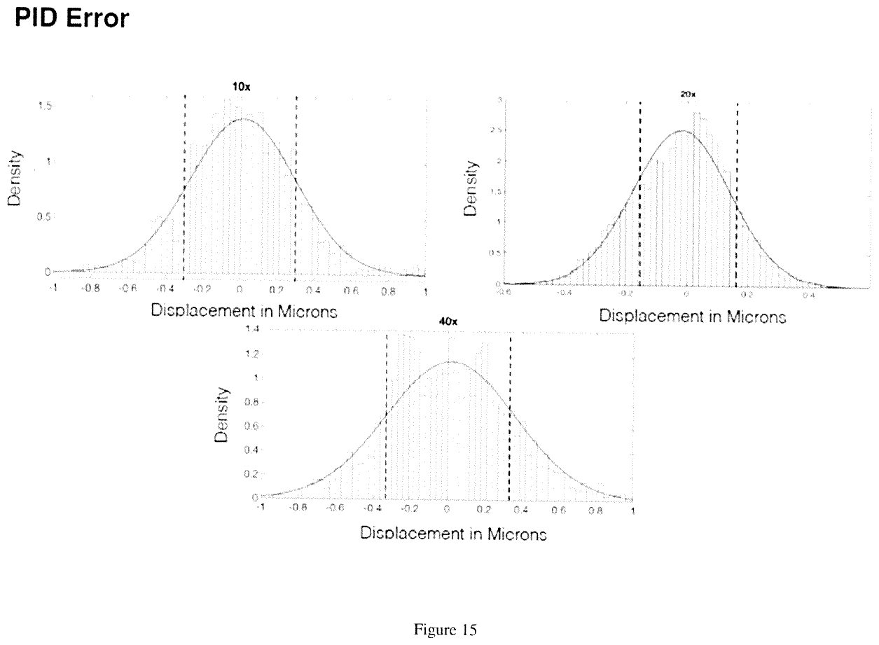

To analyze both the error and piezo position signals, a NI USB-6002 was spliced into the signal processing network. The camera trigger was also captured so that the data could be filtered down to just the data during the exposure time of the camera. If only one analog input is being acquired, the NI DAQ can acquire at a maximum sample rate of 50 kHz. However there were three analog signals acquired so that gave a maximum sample rate of 15 kHz. If the camera exposure time is 500 micro seconds, then the interesting data points would be eight data points past the rise in trigger. Using custom Matlab code, the eight data points were averaged and a data set of 210 averaged data points was formed for each run. Then, in a similar manner to the logging of camera frames, this is appended to each frame. FIG. 15 shows the histograms developed after 20 loops for each magnification. The feedback was able to keep the standard deviation within these ranges. Because the same sensitivity is achieved for each magnification, the standard deviation is comparable for each.

The next Example shows the use of this novel HSLCI system to evaluate mast cell degranulation.

### Example 4. Evaluation of Mast Cells Using HSLCI

Mast cells are a type of white blood cell, specifically, a type of granulocyte derived from the myeloid stem cell that is a part of the immune and neuroimmune systems. Although best known for their role in allergy and anaphylaxis, mast cells play an important protective role as well, being intimately involved in wound healing, angiogenesis, immune tolerance, defense against pathogens, and blood-brain barrier function. In fact, mast cells are a front line defense against pathogens and allergens in the body. Via a process known as degranulation, mast cells trigger inflammation and increases in blood flow and vascular permeability.

Degranulation is a cellular process that releases antimicrobial cytotoxic or other molecules from secretory vesicles (granule) within mast cells. The trigger for degranulation is the interaction between an antigen (e.g. from a pathogen) and the high affinity Fc receptors of IgE molecules on the mast cell surface, which activates tyrosine kinases within the cell. Subsequent degranulation releases a mixture of compounds, including e.g. histamine, heparin, proteoglycans, serotonin, and serine proteases.

While serving as a valuable immune defense against pathogens, a spectrum of mast cell activation disorders are known and range from being mildly annoying to life threatening. These disorders are unrelated to pathogenic infection and involve similar symptoms that arise from secreted mast cell intermediates, but differ slightly in their pathophysiology, treatment approach, and distinguishing symptoms. The disorder include but are not limited to: asthma, eczema, itch (from various causes), urticaria (hives), allergic rhinitis, allergic conjunctivitis, anaphylaxis (a severe systemic reaction to allergens, such as nuts, bee stings, or drugs), various autoimmune disorders such as autoimmune, inflammatory disorders of the joints (e.g., rheumatoid arthritis) and skin (e.g., bullous pemphigoid). Excessive, damaging quantities of degranulation products are also secreted in mast cell activation syndrome (mastocytosis) and in some neoplastic disorders, e.g. mastocytomas (mast cell tumors), mast cell sarcoma and mast cell leukemia.

Some agents are available to treat mast cell disorders; however, many do not work as well needed and/or they have unwanted side effects. It would be helpful to have available techniques for rapid, accurate and highly detailed analyses of candidate compounds to address this pressing need.

HSLCI provides such a technique. As shown in this Example, the use of HSLCI enabled elucidation of the amount of mass change of mast cells during degranulation (10-50% per cell), the period of time over which degranulation occurs (usually about 15 minutes) and the time period over which healthy primary mast cells recover from degranulation (24-48 hours). These mast cell properties were not previously known, and this knowledge enables detailed, high precision HSLCI screening of agents that block or modify degranulation behavior. Agents identified e.g. as inhibiting mast cell degranulation are used to treat mast cell disorders.

The new autofocus system was validated by working with primary mast cells derived from Black 6 mice. The prior art configuration was able to successfully show a decrease in mass, during degranulation, of 5-30% on a single cell basis. This gave a good baseline in determining the effectiveness of the new system. The next step in the current collaboration was to see how long it took for the cells to regain their mass. The current theory is that it takes roughly 48 to 72 hours after they degranulate. This proved problematic to measure using the 4×4 imaging grid because a mast cell does not stick to the surface of the well and can be lost after a few frames of imaging. It was predicted that the increase in imaging grid and increase in sampling wells would give enough cells to average as the cells swept across each frame from loop to loop. This approach was successful, as described below.

An advantage of high speed LCI for mast cell measurements is the ability to acquire large amounts of data in a useful amount of time. On average, 10-15 cells are found in each frame at 40×. Therefore with the HSLCI, if we capture almost nine times the amount of frames for each the control and treated samples we will get enough cells to develop a good distribution of masses.

5×10⁵ mL⁻¹ cells, media, and 200 mM dinitrophenylated human serum albumin (DNPHSA) the triggering chemical, were used in each run. Each run involved one column of six wells, with a reference well on the opposite end of a 24 well plate. For each run the control and treated wells were alternated to keep any traits inherent to the HSLCI from affecting the data. Once the cells were plated, they were carried to the HSLCI incubator that was already at the desired CO₂ and temperature levels. After 30 minutes of resting time for the cells, the experiment was started. For the first hour, or 30 runs, the HSLCI ran as fast as possible to obtain a good baseline. Next, 12.5 μL of DNP-HSA was added to the designated treated wells and the system was immediately put into acquisition mode. For the next hour, the HSLCI was run at the same time resolution so that the degranulation process could be captured. Once the second hour was finished, the system slowed the acquisition to a new frame every 20 minutes for the next 48 hours. During this time, the incubators temperature and CO₂ levels were closely monitored to insure stability.

The design of the imaging grid proved useful when averaging the large number of different mast cells. An average of 700 cells were captured per well for each loop. The imaging grid created a scanning strip down the center of each well. Because each 24 well plate is roughly 100 μm higher in the center, the cells in each well moved in synchrony where the direction was determined by the gradient at the bottom of each well. This is dictated by the wells location relative to the center, i.e. the cells eventually line the outer edge of the plate. Because of this, the average mass measurement for each well would decline exponentially after approximately 5 hours. To remedy this, the wells were stirred, using a 1000 μL pipet that was replaced for each well to avoid any cross contamination. The cells were pipetted twice, while moving around the well to insure that an even distribution was re-established. This proved to be the only step necessary; the average mass measurements fall back into line with the previously made track. This regaining of measurement stability allows omission of the measurements in which the mass is declining.

Because a large distribution of cells are seen for each loop in each well, there were small fluctuations in the mass measurements. This is remedied by calculating the difference between treated and non-treated wells. After 48 hours there was no difference in treated and non-treated cells. This is not the time that the treated cells took to regain lost mass, but the time it took for the treated cells to catch up with the untreated cells.

During the initial hour, all of the wells gained approximately 5% mass. From that point on, the non-treated wells continued to gain another 10% over the next 48 hours. For quality control, it was useful to determine the effects that focus position had on the mass measurements. Multiple metrics were plotted while a 20 μm ramp was performed. The contrast metric seen was determined using a filter that determines the overall contrast of the image by comparing the difference in neighboring pixels. The objective position that gives the least contrast is the position of best focus and was found at approximately 47 μm. When compared to the remaining metrics, a definite correlation was observed. In this particular frame it was observed that, while in focus, six cells were trackable. Within a range of 5 μm, all cells are seen with little variation in mass. This range is much larger than the range seen in where the standard deviation of the error signal, during an entire experiment at 40×, is 0.34 μm.

In conclusion, it has been shown that the low-cost addition of a laser autofocus to an existing live cell interferometric measurement apparatus allows increases in both data precision and time resolution.

While the invention has been described in terms of its several exemplary embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method for predicting the risk of and prophylactically treating graft-versus-host disease (GVHD) in a subject in need thereof, comprising the steps of:
measuring the cell biomass of CD3+ T cells isolated from a biological sample obtained from said subject;
comparing the measured cell biomass to a reference value obtained from a control subject not at risk for GVHD;
determining that said subject is at risk for developing GVHD when the measured cell biomass is higher than the reference value; and
administering an immunosuppressant therapy to said subject determined to be at risk for developing GVHD.

2. The method of claim 1, wherein said subject has had a stem cell transplantation.

3. The method of claim 2, wherein said control subject is a transplant donor.

4. The method of claim 1, wherein said measuring step is performed using live-cell interferometry (LCI).

5. The method of claim 4, wherein said LCI is high speed LCI (HSLCI).

6. A method of detecting resistance to at least one anticancer drug in a subject in need thereof and treating the subject accordingly, comprising
i) administering the at least one anticancer drug to the subject;
ii) performing HSLCI to measure a cell biomass value of tumor cells isolated from a biological sample obtained from the subject;
iii) comparing the tumor cell biomass value to a reference tumor cell biomass value obtained from a control group of tumor cells that are not resistant to the at least one anticancer drug;
iv) determining that the subject is resistant to the at least one anticancer drug when the cell biomass value is greater than the reference cell biomass value or determining that the subject is not resistant to the at least one anticancer drug when the cell biomass value is less than or equal to the reference cell biomass value; and
v) discontinuing administration of the at least one anticancer drug to the subject when the subject is determined in step iv) to be resistant to the at least one anticancer drug or continuing administration of the at least one anticancer drug to the subject when the subject is determined in step iv) to not be resistant to the at least one anticancer drug.

7. The method of claim 6, further comprising, after the step of discontinuing, administering at least one different anticancer drug to the subject.

8. The method of claim 6, wherein said biological sample is a blood sample.

9. The method of claim 6, wherein said anticancer drug is a kinase inhibitor or an immunotherapeutic agent.

10. A method of detecting resistance to at least one melanoma drug in a subject in need thereof and treating the subject accordingly, comprising
i) administering the at least one melanoma drug to the subject;
ii) measuring a cell biomass value of tumor cells isolated from a biological sample obtained from the subject;
iii) comparing the tumor cell biomass value to a reference tumor cell biomass value obtained from a control group of tumor cells that are not resistant to the at least one melanoma drug;
iv) determining that the subject is resistant to the at least one melanoma drug when the cell biomass value is greater than the reference cell biomass value or
determining that the subject is not resistant to the at least one melanoma drug when the cell biomass value is less than or equal to the reference cell biomass value; and
v) discontinuing administration of the at least one melanoma drug to the subject when the subject is determined in step iv) to be resistant to the at least one melanoma drug or
continuing administration of the at least one melanoma drug to the subject when the subject is determined in step iv) to not be resistant to the at least one melanoma drug.

11. The method of claim 10, further comprising, after the step of discontinuing, administering at least one different melanoma drug to the subject.

12. The method of claim 10, wherein said biological sample is a blood sample.

13. The method of claim 10, wherein said measuring step is performed using LCI.

14. The method of claim 13, wherein said LCI is HSLCI.

15. A method of selecting and administering one or more drugs or combination of drugs for treatment of a patient having a cancer that is resistant to one or more previously administered drugs, comprising
I) identifying a group of candidate drugs or combination of drugs that differ from the one or more previously administered drugs,
II) performing an HSLCI analysis of a biological sample having resistant cancer cells obtained from the patient to obtain a plurality of dose response curves for each candidate drug or combination of drugs in the group of candidate drugs or combination of drugs,
wherein the plurality of dose response curves is obtained at
i) a highest concentration of each candidate drug or combination of drugs equal to a peak serum concentration tolerated by patients, and
ii) one or more lower concentrations of each candidate drug or combination of drugs, each of which is lower than the highest concentration;
III) selecting for administration a drug or combination of drugs that inhibits growth of the resistant cancer cells at the lowest of the one or more lower concentrations that are tested; and
IV) administering the one or more drugs or combinations of drugs to the patient.

16. The method of claim 15, wherein said cancer is melanoma.

17. The method of claim 16, wherein said one or more drugs or combination of drugs is one or more kinase inhibitors or combination of kinase inhibitors.

18. A high-speed live-cell interferometry (HSLCI) apparatus, comprising
i) a camera with a positionable camera objective;
ii) a quantitative phase detector;
ii) a moveable observation chamber for containing a cell sample;
iii) an autofocus component configured to continuously maintain the sample within an operational depth of focus of the microscope objective,
wherein the autofocus component comprises
a) a light source;
b) a microcontroller feedback loop configured to receive a positional signal transmitted from the camera objective and calculate and transmit an offset signal;
c) a precision actuator configured to receive the offset signal from the microcontroller feedback loop and adjust a position of the microscope objective in response to the offset signal; and
d) an electronic system configured to coordinate motion of the moveable observation chamber and light emitted from the light source; and
iv) a processor configured to process phase images obtained from the camera.

19. The method of claim 18, wherein the light source is a pulsed light source.

20. The method of claim 19, wherein the pulsed light source is an electronically gated LED, an electronically modulated laser, or a flash lamp.

21. The method of claim 18, wherein the light source is an optical fiber.

22. The method of claim 18, wherein the light source is a laser.

23. The method of claim 18, wherein the cell sample comprises single isolated cells, cell clusters, or organoids.

24. The method of claim 18, wherein the cells are attached to the chamber surface, suspended in solution or within a solid transparent matrix material

25. The method of claim 24, wherein the solid transparent matrix material is gelatin.

26. The method of claim 18, wherein the autofocus component is a coaxial optical beam deflection displacement sensor.

27. The method of claim 18, wherein the precision actuator is a piezo actuator.

28. A live cell interferometric measurement apparatus, comprising:
a means to image cells in a volume;
means to compute cell mass based on images of cells; and
a laser autofocus feature for focusing the imaging means on the cells in the volume.
